SEQUENCE LISTING (14 sequences)

SEQ ID NO: 1 - DNA, 18227 bp, Xenorhabdus nematophilus
gaattgctcagtgaatggaaaagccataagcaggagttgatcgattttctgaatcgaattgatgcacaagaggacgttcaaactcacccgctacaggatattcaacgtaatggcaacgttgcacattacccactttcatatgctcagcaacgtttatggtttattgaccaattgaccgaaggcagcacccagtacaactgtgtcggggatttcagactgcgtgagccacttaatatcaagtttttcgaagcagccctgaaagggatcattgagcgtcatgaagcattgcgaactcattttaaaactataggggatgaacctcgtcaggttattgccactcactacgatttgcctgtgaaataccacgatttgtcagccttttctgatgttgagaaaaaagatcaggtgaaacgactccataaagaagaagaaaatctggcctttaacctcagcaccgatttgatgctgcgtgttcaaatattaaaactggctgaaaacgattatgttattatctataacattcaccatatcgcttgtgatagttggtcaatagaaatattcctcagagagctgattaccttatatcgggcttattgccaaggtgaagaaaacccattgccagtgctaaaggttcaatatgctgattatgcccaatggcaacgaaactggctgcaaggcaatgtcctggaaaaacagctggattactggcagacgcaattatcaggcatttcaccacttcatcattttccactggataatcctcgccctgaaaaacagagctttgaaggacgtatacatttgcagcgtatttccaaagagttgactcagggtattaaaacactatgtaccaaacatgaagtgacattatttatgtttctggaaacggcatttgctgttttactcagccgttacagcaatgaaaaagatattctggtgggaacggccctggcgggaagaacacaccaagacgtagaatctttgattggtttttttgttaactctttggtcatcagaacagatttgtccggaaaaccgacatttagtgaactattgaaacaaaacagccgcaacattttggatgcttatgcccatcaagatctgccattccaaacgctggtagaaaaaatcagcccggccagaaacttaaactataacccgatattccagatagtttttacgctggagaatacccagcatgatacgatattggaaaagaataataatattgagatagaagctcgcccatttgttaaagccagatttgatttggaaatccatatttacgaaaaagacaacgaattatctttggaatggatttcagatatcagcctgttcaacagcaatactattgacagattactcgccaattacgagaccctgctgaccaatattgttgatgtcatggaaaggagtgcaataaatgaagagccatccatctgtgaactgcctattttggctaatgcggaacaatacacattattgcatgaattaaatgggccacagactcagtatcaacgcggacgctgttttcatgaactgtttgaagagcaggttatcaatacccctgaaaaaatggcgttagtttttggtgagcatgctttaagttaccgggcattgaatgaacaggccaatcaactggctcattacctgattgaccaaggcatccgacctgatactttggttgcaatttgtcttccacgctcactacagacagttattgtactgttgggaattcttaaagcgggcggagcgtatgtacccctggatgccagttatcctaaagcccgcctgcaatacatgctggagcacagcggggcagagattattctgaccgagacatctttggtcaatcaattgcctgtcagccagcaacaggttatttgtctggacacagatacggtacagtcaaatgtaaaagcgatgtcggcagacaatattatcgaacgtaccgcgcctctgacagaaaatcatctggcctatgttatctacacctccggctcgacaggcaaacctaaaggcgcaatgctggaacacaaaggttgggttaatctggctcatgcgcaagctactttgttcggggttgatgcaaatatccgtgtattacaatttgcttcctggagctttgatgctgccattttggaaatgtctatgacattggcatatggcgccacgctgtacctcacatctgaaacacaacgtcgttcccctgaattgttgaatgaattcattgcaaaataccagattacccatgctgtattgccgcctgcgctgctaccttatttgaatttcaatgaatggcgcagtgtttcatctttactattggcgggagaagccgtaacaccgcagatttcagcccattggtcacagggtagaaaattgtttaacgtgtacggcccgacagagtgtacttcaattattaccagcggattattaaccgacgaaaaaattacgataggcaaaccgctaccgaatgctgtcatacgtattttagatagcaatgggcatttaaccccgattggtgttaccggagaattgcatatcggtggtattcagctggcccgcggttaccggaatgcgccggaactgacagaaaagcagtttatttgtgatctcttcagtcctaactcggctgacaagctttatcgtaccggcgatttagtccgttggaccctcgacggtgaattggaatttattggccgcattgattcacaggtcaaaattcgcagccatcgtattgagttaggagaaatcgaatccgttctggccggacaagacattttaagtagtgcggcggtgattgcttatagtcatgacgacaaagataaacaattggttgcctatgtttgccccagtgccgactggctggcagaaaaagccgccgcatttaatgccgattacgtgcaaagctggacagaaatctttgatgagcaataccggcagaagaatgtcaaaaatacccttgctgataagcatgacgctggcagtgattttggcggctggataaacagttataccgatcaaccgatcccgcttgaacagatggaagagtggcagacgggcaccatgcagcgaattcatgccctgcatccccgccgtttactggaaattggctgtggtaccggtttactgttgtatcaatatgctgaacagtgtgaatcggtcctggccaccgatatttcggccgaagtgctggcacggcatcagcaaatcctgcaacaacgcggttggtcacatgtccagttgcgtcagggggatgcccttaattcaggtgcattggcaccggacgcttttgatacggttatcattaactcggtggtgcaatacttccccaatgttcagtatctggacaatgttattgcccagttactcccggcggtggaagcgggcggaaaaatcctcttgggggatatccgcaacctggatttgctgacggcgcatgtgacggcaatagaacaaggccatctgggtgaacagcgcattgccgtgggaacgatggtcaaccgtattcagcgccgcttgcaacaagaagaagagtttctgatcagcccgacttattttgcccaattacccgcccgttatccggagattggccgggtggatattctggtgaagcggggcattggtgacaatgaaatgctgcgctaccgctatgaagtcatcttacataaacgggataacaaagccgtgtcatgccatgatcagcctgtcacctggtttgatttcagtgcgattgacgcggtgagcagtctgttacaggcgggaacttacgatacctttggtttttctggcctgcccaacgcccgtatcaaagaggacgctgaactggcggaaggactgcgtcattggccagccagccaaatactcttcccgtcagaatatgccggaaaattttctcctcaggcagaaaaacaggttcacgccttggaatccctgtttcagcatgcggagcagtgtggttatcaatgcggcgtgacctggtcacaaaaacaagcggactggctggatgtcattttcagtcgtgatgagctgccgcctgtacaggcccgtcatcaatacagccaaatctatttagccaattatccgcaaatttctgccattggcggggaactttcagagcgactggaaacggccctgaaaaaacaattgccggaatatatggtgcccagcctttatatcccactggagcgcatgccgctctcactgaacaataaaatcgataaaaacgcgctgccaatcccaaatgacagtgatttacgtcaacaggtttatgtcgcaccagaaaatgaaatagagattaaaatcggccagttatggcaaaggttgctgaatgtcaggcaagtgggcatccatgacaatttctttacgctgggcggacattcacttcaggcaacgaaattgatcaattcaatacgtaacgatttagatgtcgatatccctttgaaaactatttttgagcatccaaccctcgagcaattatcgacagtaataaccaatctcttcgttatcaaagaaaaaagaaaactttttcaggcagaacaagaaacaacacaagaactattggaaggtgatatatgaaacatgctgcacagattgtcagtgaagctttagagcaaggaattaccctgttcgttgtcgataaccaattaaaataccaagcaaaacgcgacagcattcctccggaactgcttagtgagtggaagaaccataaacaagagttgattgattttcttaaccaaatcgattcagattggtcacaaccaatccaacattatgaaaaggcagaacattatccgctttcatttgcccaacagcgtttgtggttgattgaccagttaaatggcagcagcccccagtacaacggaaccggggatttcagattaaaagaagccgtgaatattgaggcatttgaagcggcagtgaaaagcttgcttgaacgtcatgaagcattgcgtactcactttacagttattgataacgagccgcggcaggtgattgcgacgacctatgatctgcctatgacctatcatgatttatcggcactgtctgaaacagaaaaaaaacatcacttaaaacgactcaataaagaggaatgggatcaggtctttaacctcagtactgatttgatgctgcgtatccgagccattaaattggcagaaaatgattatgtcattatctatacgatgcaccatatcgcctacgatggctggtcgatcccgattttcctcagtgaacttctcaccttgtaccgggcttattgcaatggtgaaaataatccgctgtccccgctaaaagtccagtatgccgactatgcccaatggcaacggaactggctgcaaggcgaaattctggaaaaacaactgacgtattggcaaaaccagctagccggcattactcccttgcatcgtttcccgctggataatccccggccagaaaaacaaggtttcgaaggacacgttcattcacagcgtctttcagcacatcttacccaaaaaatcagggcactgtgtaccaggcatgaagtcactttatttatgttcctggagactgcgtttgccgttttgctcagccgctacagcaatgaaaaagatatcgtggttggcatgccaatcgccggcagaagacaccgcgatattgaacccctgatcggctttttcgtcaattctctggttatccgaacggatttatccgggcagcctactttcagtgcattgctaaaacaaaacagccgcaccattctgaatgcgtatgcacatcaagatctgccctttgaaatgttagtggaaaaaatcagcccggagcgaaatttaaaccataatcctgtcttccagatcatctttgccgtacagaacaaccagcaagatactacgctggagcaggataatattgatccggatgaagaaaagacacttcttacgacccgttttgatctggaagttcatgtttatgaagacgaagagaaaggcgggctatcgattttatggatttcagataccagcctgtttaacagagataccattgacagattgctcgctaattacgaaaccctgttgactagcattgtcgaggctatgaatgtcagtccggccaataaagagccaaccattgataaacttccactattaggtgatgtggaaaaacactcattattgcatgagttgaatggatcacagactcagtaccaacgcggtctttgtttccatgagctatttgaagaacaagttgcgctcaatcccgaaaaaaccgcattggtttttggcgaaagcacgctcagttatagagaattgaatgagcaggccaaccaattggcgcattacctgattgaacagggtatccagcctgataccttggttgcaatttgtcttccgcggacgctgcaaatggctgtcacactactgggtatcctaaaagccgggggggcttatgtcccgttggatatcagctatccgaaagcgcgtcttcaatatatgctggaacacagcggggcggaatttattctgactgaaacgcatttagtcgataacctgccactcaatcaacaaaatgtcatttgtttggataccagcgaaatacaatcacatctaaaaaatttaccaacagaaaacattatcgaacgcccattcccactgactgaaaaccatttggcctatgtgatttatacctctggctccacaggcagaccaaaaggcgcaatgctggaacacaaaggctgggttaatctggcacacgcgcaggctgcgttatttggggctgattcaaatatccgcggattacaatttgcctcctggagttttgatgctgccattctggaaatgtccatgacattggcatacggggcaacactgtatcttatttctgaaacccagcgccgttcccctgagttactggatgccatcgttgaaaaatatcagattactcatgccgtattaccgccagcgttattacctcatctggatttcagcaaatggcgcagtgtttcaaccctgttgttagccggggaggctgtgccaccccatattgccgctcaatggtcacaagagcggaacttgttcaacgtttatggcccgacagaatgtacctctattatcacctgcggattattaacggcagataagcggatcacgattggcaaaccactgccgaatgccgtgatgcgcattatggactccgatggcaatttagtcccgcttggtgcggagggggaactgcatatcggcggtatccagttggcacgcggttaccggaatgcgccagaactgacagaaaagcagtttatttgtgatcccttcagccctaacccgactgacaagctttatcgtaccggcgatttggtgcgctggacccctgaaggtgaattggattttattggccgtattgattcacaggtcaaaattcgcagccatcgaatcgagctgggtgagattgagtccgttctggcgggacaggatattttaagcagtgcggcagtgatcacaaatggtgacagtgatgataagaaactgattgcttatgtttgtccaagtacggattggttggcagaaaaagccacagcttttaacgctgactatcttgaaagctggacaaaaatctttgatgaccaatatcgacagacagcgactgaaaacgttatttccgataaacatgacactgacagtgattttggcggctggataaacagttataccaatcagccgatcccgcttgagcaaatggaagagtggcgtgcgggtaccatgcaacgaattgacctcctgcatccccgtcgtttactggaaatcggttgtggtacaggtttactgttatatcgctatgcggaacagtgtgaatcggtattggcgacggatatttcggcagaagtactggcgcgacatcagcaaatcctgcaacaacgcggttggtcgcatgtccagttacgtcagggggatgcgctgaatttgggtatgatcacgcccggcatgttcgatacggttgtcattaactcggtggttcagtatttccccaatgtccagtatctggacaatgttattgctcagttactgcctgcggttgaagcaagtgggaaaattctgctgggggatatccgcaaccttgatctgctgacagcacatgtgacggcgatagaacaaagccatctgggtgaacagcgtatttccgtgggaacactggcaaaccgtattcaaagacatttacagcaagaagaagagttcctgctcagcccgacttattttgctcaactgccaacacgttacccagagatcggcagagtcgatattctggtcaaacgtggcgtaggcgataatgagatgctgcgttatcgttatgacgtcatactgcataaaaaaggtgaaaatgcacaatcttgtggtcatgctcctttcacttggcatgattttgtttcccttgaaaatttacgtgatatgttacaaagggaagagcaaacattcggtgtttcgggtatccccaatgcccgcgttaaagacgatcttgccttggctgaggggttacgccattggccggccaatcaatttatttcttcatctgaacaagcgggtaatttctcctcgcaatcagtggaacagattcaatctcttgaattactgctccaatatgctgaacagtgcggttatcagtgtggtatgacgtggtcacaacaacaacctgatctgctggatgtgattttcagccgtggaacattgcctcaaatacaggctcgttctgattacagtcaaacccatctggctaattatccgcaaatttcgtctattagtggtgaactttcagaactccttgaatctgcgctgaaaaagcaattgccggaatatatggtgcccagcctgtatatccccctggagcgtatgccactctctctgaacaataaaatcgataaaaaagcactgccggttcctaacgaggatgatttgcggcggcaggcttataccgctccaagagatgaaactgagaagaagctttgtcagttatggaaaaatttattgaaagttcatcaggttgggattcatgacaatttcttcacactgggcgggcattcacttcaggcaacacgtttgattagctcaatacgcagtgaattagacgttgaaattccattaagaactatttttgagcacccgacactggagcaattatcaaaagttgtcacagttcatcttgtcatggcaagaagaaaacatttccagacagagcaagggacaacacaaaaattattgaaaggtgatatatgaaaaacgccgctcagattgtcaatgaagctttaagtcagggaattaccctgtttgttgctgataatcgcttacaatatgaaactcagtgcgacattataccgccggaattgcttaatgaatggaaagaacataaacatgaattgattaagtttctgagtgacgccaattcagacgaggaaacacacacctatcaattgcaagctatccaacgtgatgagcatgcaaccgattacccactctcatttgcccaacagcgtttgtggttgattgatcaggctaatggcagcagcccccagtacaacggtaccggggatttcagattaaaagaaaccgtgaatattgaggcacttgaagcggcagtgaagagcttgcttgaacgtcatgaagcattgcgcactcattttaaaatcattgataacgaaccgcggcaggtgattgcgacaacttacaatctgcccgttatttatcatgatttatcggcactgtctgaaacagacaaaaaccatcacttaaaacgactcaataaagaagaaggtgatcagatttttaacctcagtaccgatctaatgctgcgtatccgggtcgttaaattggccgaaaatgattacgttattatctatacgatgcaccatatcgcctacgatggctggtcgatcccgattttcctcagtgaatttttcaccttatatagggcttattgccacggtgaaaataatccgctacccccgctaaagatccaatatgccgactatgcccgatggcaacggaactggctgcaaggcgaaattctggaaaaacaactgacgtattggcaaaaccagttagctggcattgtccctttacaccattttccgttggataatccccggccggaaaaacagggcttcgaagggcgtgttcattcgcaatgtttttcagagcatttcactcaaaaaatcagggcactgtgcgccaaacatgaagtcactttatttatgtttctggaaacggcgtttgccgttttgctcagccgctacagcaatgaaaaagatatcgtgattggcatgccaatcgctggcagaagacaccgtgatattgagtccctgatcggctttttcgtcaattctctggttatccgaacggatctatccgggcagcctactttcagtgaattattaaagcaaaacagccgcaccattctggatgcttatgagtatcaagatctgccctttgagatgttggtggaaaaactcagcccggagcgaaatttaaatcataatcctgtcttccagatcatgtttgccgtacagaacaatcaacaagattttatttgggagcaggaacatattgttcaagatgaagaaaaaccgcttctcacgacccgttttgatttggaagtccatgtttatgaacaggagaatgagttatgggttgtatggattgcagataccagtctgtttaacagcgataccattgacagattactcgctaattacgaaaccctgctgaccagcattgttgaggcgatgagcgaccattcggttaataatgagccatccgtccatgatctcccgtttctggctgatgcagaaaaacatatgctactgcatgagttgaatgggccacaggcccagtatcaacgcgatctgtgtttccatgagttgtttgaagagcaagttgcactttatcctgaaaaaaacgcgttgatttttggcgaaagcacgctcagttatagagaattgaatgagcaggctaaccaattggcgcactatcttattgaactgggtattcagcccgataccttgattgcgatttgccttcctcggtcattacaggcagtggttgtactgttgggtatccttaaagctggcggggcttatgtgccgctggatgccagttatccgaaagcgcgtcttcaatatatgctggagcacagtggggcggaatttattctgactgaaacatctttggtcgaaaaattacctgtcagccaacaaaaagttatttgtctggacagtgacgaaatacagtcacatgtgcagaacatgctggcagacaatattgttgatcgacttttgcccctggcagaaaatcatctggcctatgttatctacacctccggttcgacaggcaggccaaagggcgcgatgatggaacacaagggttgggtcaatctggctcatgcgcaaaccacgttgtttgggattgatacagatagtcgtgtattacaatttgcttcctggagttttgatgctgccattctggaaatgtccatgacattggcatacggggcaacactgtatcttatttctgaaacccagcgacgttcccctgagttactggatgcaatcattgaaaaatctcagattacccatgccgtattaccaccagcattattacctcatttgggtttcagtaagtggcgcagcgtttcaactctgctattagccggggaggccgtaccaccgcatattgctgcacagtggtcacaagatcggaaattgttcaatgtctatggtccgacagagtgtacctccattatcacctgcggattattaacagcagataagcggatcacgattggcaaaccactgccgaatgccgtgatgcgcattatggactccgatggcaatttagtcccatttggtgcagaaggggagctgcatatcggcggtattcaactggcccgcggttaccggaatgcgccggaactgacagaaaagcagtttatccgtgatcccttcagtactaacccggctgacaagctttatcgtaccggtgatttagtccgctggaccctcgatggtgaattggaatttattggccgcattgattcacaggtcaaaattcgcagccatcgaatcgagctgggtgagattgagtccgttctggccggacaggacattttaagcagtgcagtagtgatcacaaatggtgacagtgatgataaaaaactgattgcttatgtttgccccagtgcagagtggctggctagaaaagctaccgaatttaatgccgattatgtgcaaagctggacagaaatttttgatgagcaataccgacagaaaagtgtcaaaaataccctcgctgatcagcatgatgcggatagtgattttggcggttggataaacagttataccgatcaacccatcccgcttgaacagatggaagagtggcggacgggcaccatgcaacgaattaatgccctgcatccccgccatttactggaaattggctgtggttctggtctcctgttatatcgctatgcggaacagtgtgaatcagtcctggccaccgatatctcagccgaagtgctggcgcggcatcagcacagcctgcaacaacgcggttggtcacatgtccagttgcgtcagggagatgcccttaattcgggggtattggcaccggacacttttgataccgttatcattaactcggtggtgcaatacttccccaatgttcagtatctggacaatgtccttgcccagttattgccggcggttgccgcagggggaaaaattttgctgggggatatccgcaacttagatctgctgacaacacatgtgacggcgatagaacagagccatctgggtgaacagcgcatttccgtgagcacgatggccaaccgtattcagcggcgcttgcagcaagaagaagagttcctgatcagcccgacctatttcgctcaattacccgcccgttatccggagattagccgggtggatattctggttaaacggggcgtcggtgacaatgaaatgctgcgctatcgctatgaagtcatcttgcataaacgggataaccatgctgcatcatgtcacgatcggcctgtcacctggtttgatttcaatacgattgacgcggtgagccgtctgctgcaagcaggaacttacaagacctttggtatttcaggtatgcccaacgcccgtatcaaagaggatgttgaattggctgaaggactgcgtcattggccggccaatcaaatactatcttcactgaaaaataccggctgtttgacctcggaagcgatggagcaaattttaactttcgaatccttgcttcagtatgcggagcagtgcggttatcaatgcggcgtgacgtggtcacaaaaacaatcggacttgttggatgtgattttcggtcgtgatgagttgcctcctgtccaggcccgtcatcaatacagccaaacccatttagccaattatccgcaaatctctgctattggaggggaactttcagagcaactggaaatggccctgaaaaaacagttgcctgactatatggtgcccagcctttatatcccactggagcgtatgccactctcgctgaacaataaaattgataaaaaagcgctgccggttccagatgacagtgatgtacaccatcaaaattatgtcgcccccagagatgaaattgaggaaaaactctgtcagttgtggcaaaagcatctgaaaatcaatcaagtgggtatttatgataatttcttctcactcggtggccattcgctcttggcagtgaaaataacagcctccataaaaaatgatttaagtaacaatttcagtatgaatcaaatgttttcgaatccgactattgcacagattgccagtgtgattcgtagcgctcaagaccatgacatatctacactattgccaacacaacgtgtatccgacggaaaaataccaatttcttattaccaaaagatgttctggtctctgatccaagaaagagtattggacgatgcctttaatgttcccattaccttgttgatggagggcgaattaaaccaaagtgcattggaaaaaagtttgactgttattttagaacgacatgaaagtttacgattaagattttatgaagaaaatggacagtttttcatgaaaccgaataacagcatgaaaatcaatttacaaattatcgattcgttgccaaaaggaatggagaaagaacaaattaccattgaaattaacaaactctgggaagagaaattaagagtaccttttgatatttacaatggcccattattcaagacatttttgctgcctgtatcagaaacacgccatgtcttattagttaatattcatcatattatctgtgatggatggtctatcaatatttttatgaatgaatttaagaaactatactcggcttactcacaggaacaagatattattcttcccatagttgcaatgcaatattctgactgtgtttatgatttgcaagaacgacataagactgaaaaatatcaaaggcagctaaaattctggctgcaacaatttgctgatattgatatgaatgaacgttgttttccagtcgctttcagcaaacatctgactactgagtatcaatttaaagcgattaatattaatactccaaaatcattggataaggctgctgtacaatattgcgaaatccataagattacaccttatatgttatatatggctttgttccacgcttgtctttttatacattctggtgaatcacaacatatcgccacatcaccacaagctgatcgttctcatattgacagccatcaaacaataggactgtttttgagtgacctgatagttaaatcaactatcagtaaagagatgagcctttatgaaatcattcagcaagtcagtgattttatttataaagcgctggaaaattcagatatccatatggctgcggtgattgataacgtaggggaaaaggtgagagataatatatttaatattgtctttaactatatggatgctcagaatctctatgattatgcagataattctgatagtggaaaaatatccctgccaagcctcgatatagaattgataaatattgacccgactccttttcaatcactggggataaacttcatcaacataccacaaaatatcatgtgcgagttaagctataacccagagctgtatactgaagaaactgccaataatcttgcaagatattatggacgactgctggaagccattgtctctggtcaagataaagaaaatatcagttacttttatgactaaattcatttaataaataatccgttaatgaataaaaagtcaggcctcatgaatgaggccaattattgttaatcgtagtcaatttactggcgtaattttacttgggatattaatgagttcaatatcaggtttatattccgaatctatcaaaatataatcacagccatattcattgcacattttgaaattgtattcattttcttcaatcgttgattcaagtgtacaatccgaatcatctaaccgagattcaatggcatttgcatatttttttaacatccgaaaagtgattttcaatatactctttagtcatcacaagacagtgaaattggatttgcctcagatagttatctgtgaaagcatctttccagtcaaaaggaatgtaacttccttcaataacaagattctgattattctcaattgctgtcttgatgatctccttaacaatcggccaaagatacgattcaagtttcttatcagattcaagtttcttatcatcttccggggttataccaaagccctattattctggataatgcgggttacccccgggcagaatgcgtgaaagatattgcgtatgggcgtaatattgaattgcattacctgccgcctgacagcccaaatctcaatccgatagagcgattgtggaagtatatgaatgagccagtgcgtaacaatgtctattttccggatgcgcagacgtttcgtgaaaccctgcgtcactttttccatgtcatgttgccagaaaaaacgaaagaactcacgacccgactgactgaccattttcatttactatgtgaatctgtttttcaggtaatatctcattacctcttatttcatggaaaatctccattccaaacaggggataattaaaaatatttaaaggtgatatatgaaaaatgcaattcaaattattaatgaagctttggaatatggcattacgctatctattgagaatggaaaattacaatatgaaactgattgtgatattattccatctcaattgtcatatgaatggattaataacaaaaatcaattaatgagctttctggaactgtcttatccaacagaaaacatgtcgcaaggcaatattctggataatcaattggattattgggaaaaacaactagctagcatctctccgctgcatcattttccattggataacccccgttcagaaaaacagagtttcgaaaaatgtacacatcaacaacgtatttcaaataatcttatacaagaaataaaaacgttatgcacccaacatgaagtcactttatttacgtttttagagactgcctttgctgtgttgtttagccattacagtaatgaaaaagatattgtaattggaacactaattacaaaaagaaaacgctatgatattgactcttcaatgggatactttgtcaattcactggttatcagaacagatttatcctctcagcctactttcagtgaattattaaaacaaaatagtcatactatcttggatgcttatgcacaccaagaacttccttttgaaagattggcaaaaaaaataagccctagtcataatctaaactatcatcctattttccaaattgcgtttacgctaggaaagactgatcagaaaaatatggctttggcgcaggaaaattacgttaatagtggagaatacctgcatctgaaaaccctgcttgatttagaaatccacattgatgaaaaagatggggaagaattatctcttttctggaattatgatactgatttgtttgaaaataccacgattgaaagattaagtgccaattatgaaaccctactttccaatattgttgcaatgatgacgcaggatgttgatgaagaactgcctgctcaggaaccctctgttcataacattccatttctatctgatactgaaaagtacactttattgcatgaatggaatgggccacagaaacattatcaaacagggcgttgtttccatgacttgtttgaagaacaagctgaacgagtgcctgaaaataccgcactggtttttggtgatgattcactgacttatcgagcagtcaatgaacaagccaaccaactggcacattatttgatagaattgggtgtcaaacctgatacgctggtcgcactttgtgtgtcgagatcattacaagggatagtagcgttactgggaattcttaaagcaggcggtgcatatgtaccgttagatcccggttacccgcaggcacgccttcagtatatgctggagcatagtgaagttgagttcatccttaccgaaacacatctggcagaaaaattgcctatccgccagcaaacagtgatctatctggatacacagaccgtacaattacacctgcaaaatatgccaaccgggaatatcaccagacgctcaatgcctctgacagaagataatctgatttatgtgatttatacttctggctctacgggcaaacccaaaggcgtaatggtgaagcataaaggctgggtcaatctggcattatctcaggccgatctatttgggattgatgcaaacagccgggtattacagtttgcttcatggagctttgtggcaatgaccatagagatggcgatggctttcccttgtggcgcaacgctgtgtttgatttcagaaacgcagcaacgtactcctgagctactggatgatatggttgaaaaatatcagattactcatactatgctgccgcaagcgctattacctcatctgaacttcaacaaatggcaatccgtctcaaatttattattggggggagaggctgtaccaccacaaatagcaactcgttggaaaccgggcagaaaactatttaacgcatatggctctacagaaacttcctctatggtaactgctggtttattaattgatgaccgaattacaattggggaaaccattgcctaatgtagtggttcgtattcttgatccggagaggagtctggtaccaattggggcaattggggaactgcatatcggtggggttcagttggcacggggttaccggaattc

SEQ ID NO: 2 - DNA, 4683 bp, Xenorhabdus nematophilus
atgaaaaaggccgtgcaaattatcaatgaggcgttaaatcagggaattaccctgtttgttgttgataatcaattacaatatgaaaccaaccgcgacagcataccgccagaattgctcagtgaatggaaaagccataagcaggagttgatcgattttctgaatcgaattgatgcacaagaggacgttcaaactcacccgctacaggatattcaacgtaatggcaacgttgcacattacccactttcatatgctcagcaacgtttatggtttattgaccaattgaccgaaggcagcacccagtacaactgtgtcggggatttcagactgcgtgagccacttaatatcaagtttttcgaagcagccctgaaagggatcattgagcgtcatgaagcattgcgaactcattttaaaactataggggatgaacctcgtcaggttattgccactcactacgatttgcctgtgaaataccacgatttgtcagccttttctgatgttgagaaaaaagatcaggtgaaacgactccataaagaagaagaaaatctggcctttaacctcagcaccgatttgatgctgcgtgttcaaatattaaaactggctgaaaacgattatgttattatctataacattcaccatatcgcttgtgatagttggtcaatagaaatattcctcagagagctgattaccttatatcgggcttattgccaaggtgaagaaaacccattgccagtgctaaaggttcaatatgctgattatgcccaatggcaacgaaactggctgcaaggcaatgtcctggaaaaacagctggattactggcagacgcaattatcaggcatttcaccacttcatcattttccactggataatcctcgccctgaaaaacagagctttgaaggacgtatacatttgcagcgtatttccaaagagttgactcagggtattaaaacactatgtaccaaacatgaagtgacattatttatgtttctggaaacggcatttgctgttttactcagccgttacagcaatgaaaaagatattctggtgggaacggccctggcgggaagaacacaccaagacgtagaatctttgattggtttttttgttaactctttggtcatcagaacagatttgtccggaaaaccgacatttagtgaactattgaaacaaaacagccgcaacattttggatgcttatgcccatcaagatctgccattccaaacgctggtagaaaaaatcagcccggccagaaacttaaactataacccgatattccagatagtttttacgctggagaatacccagcatgatacgatattggaaaagaataataatattgagatagaagctcgcccatttgttaaagccagatttgatttggaaatccatatttacgaaaaagacaacgaattatctttggaatggatttcagatatcagcctgttcaacagcaatactattgacagattactcgccaattacgagaccctgctgaccaatattgttgatgtcatggaaaggagtgcaataaatgaagagccatccatctgtgaactgcctattttggctaatgcggaacaatacacattattgcatgaattaaatgggccacagactcagtatcaacgcggacgctgttttcatgaactgtttgaagagcaggttatcaatacccctgaaaaaatggcgttagtttttggtgagcatgctttaagttaccgggcattgaatgaacaggccaatcaactggctcattacctgattgaccaaggcatccgacctgatactttggttgcaatttgtcttccacgctcactacagacagttattgtactgttgggaattcttaaagcgggcggagcgtatgtacccctggatgccagttatcctaaagcccgcctgcaatacatgctggagcacagcggggcagagattattctgaccgagacatctttggtcaatcaattgcctgtcagccagcaacaggttatttgtctggacacagatacggtacagtcaaatgtaaaagcgatgtcggcagacaatattatcgaacgtaccgcgcctctgacagaaaatcatctggcctatgttatctacacctccggctcgacaggcaaacctaaaggcgcaatgctggaacacaaaggttgggttaatctggctcatgcgcaagctactttgttcggggttgatgcaaatatccgtgtattacaatttgcttcctggagctttgatgctgccattttggaaatgtctatgacattggcatatggcgccacgctgtacctcacatctgaaacacaacgtcgttcccctgaattgttgaatgaattcattgcaaaataccagattacccatgctgtattgccgcctgcgctgctaccttatttgaatttcaatgaatggcgcagtgtttcatctttactattggcgggagaagccgtaacaccgcagatttcagcccattggtcacagggtagaaaattgtttaacgtgtacggcccgacagagtgtacttcaattattaccagcggattattaaccgacgaaaaaattacgataggcaaaccgctaccgaatgctgtcatacgtattttagatagcaatgggcatttaaccccgattggtgttaccggagaattgcatatcggtggtattcagctggcccgcggttaccggaatgcgccggaactgacagaaaagcagtttatttgtgatctcttcagtcctaactcggctgacaagctttatcgtaccggcgatttagtccgttggaccctcgacggtgaattggaatttattggccgcattgattcacaggtcaaaattcgcagccatcgtattgagttaggagaaatcgaatccgttctggccggacaagacattttaagtagtgcggcggtgattgcttatagtcatgacgacaaagataaacaattggttgcctatgtttgccccagtgccgactggctggcagaaaaagccgccgcatttaatgccgattacgtgcaaagctggacagaaatctttgatgagcaataccggcagaagaatgtcaaaaatacccttgctgataagcatgacgctggcagtgattttggcggctggataaacagttataccgatcaaccgatcccgcttgaacagatggaagagtggcagacgggcaccatgcagcgaattcatgccctgcatccccgccgtttactggaaattggctgtggtaccggtttactgttgtatcaatatgctgaacagtgtgaatcggtcctggccaccgatatttcggccgaagtgctggcacggcatcagcaaatcctgcaacaacgcggttggtcacatgtccagttgcgtcagggggatgcccttaattcaggtgcattggcaccggacgcttttgatacggttatcattaactcggtggtgcaatacttccccaatgttcagtatctggacaatgttattgcccagttactcccggcggtggaagcgggcggaaaaatcctcttgggggatatccgcaacctggatttgctgacggcgcatgtgacggcaatagaacaaggccatctgggtgaacagcgcattgccgtgggaacgatggtcaaccgtattcagcgccgcttgcaacaagaagaagagtttctgatcagcccgacttattttgcccaattacccgcccgttatccggagattggccgggtggatattctggtgaagcggggcattggtgacaatgaaatgctgcgctaccgctatgaagtcatcttacataaacgggataacaaagccgtgtcatgccatgatcagcctgtcacctggtttgatttcagtgcgattgacgcggtgagcagtctgttacaggcgggaacttacgatacctttggtttttctggcctgcccaacgcccgtatcaaagaggacgctgaactggcggaaggactgcgtcattggccagccagccaaatactcttcccgtcagaatatgccggaaaattttctcctcaggcagaaaaacaggttcacgccttggaatccctgtttcagcatgcggagcagtgtggttatcaatgcggcgtgacctggtcacaaaaacaagcggactggctggatgtcattttcagtcgtgatgagctgccgcctgtacaggcccgtcatcaatacagccaaatctatttagccaattatccgcaaatttctgccattggcggggaactttcagagcgactggaaacggccctgaaaaaacaattgccggaatatatggtgcccagcctttatatcccactggagcgcatgccgctctcactgaacaataaaatcgataaaaacgcgctgccaatcccaaatgacagtgatttacgtcaacaggtttatgtcgcaccagaaaatgaaatagagattaaaatcggccagttatggcaaaggttgctgaatgtcaggcaagtgggcatccatgacaatttctttacgctgggcggacattcacttcaggcaacgaaattgatcaattcaatacgtaacgatttagatgtcgatatccctttgaaaactatttttgagcatccaaccctcgagcaattatcgacagtaataaccaatctcttcgttatcaaagaaaaaagaaaactttttcaggcagaacaagaaacaacacaagaactattggaaggtgatatatga

SEQ ID NO: 3 - DNA, 4665 bp, Xenorhabdus nematophilus
atgaaacatgctgcacagattgtcagtgaagctttagagcaaggaattaccctgttcgttgtcgataaccaattaaaataccaagcaaaacgcgacagcattcctccggaactgcttagtgagtggaagaaccataaacaagagttgattgattttcttaaccaaatcgattcagattggtcacaaccaatccaacattatgaaaaggcagaacattatccgctttcatttgcccaacagcgtttgtggttgattgaccagttaaatggcagcagcccccagtacaacggaaccggggatttcagattaaaagaagccgtgaatattgaggcatttgaagcggcagtgaaaagcttgcttgaacgtcatgaagcattgcgtactcactttacagttattgataacgagccgcggcaggtgattgcgacgacctatgatctgcctatgacctatcatgatttatcggcactgtctgaaacagaaaaaaaacatcacttaaaacgactcaataaagaggaatgggatcaggtctttaacctcagtactgatttgatgctgcgtatccgagccattaaattggcagaaaatgattatgtcattatctatacgatgcaccatatcgcctacgatggctggtcgatcccgattttcctcagtgaacttctcaccttgtaccgggcttattgcaatggtgaaaataatccgctgtccccgctaaaagtccagtatgccgactatgcccaatggcaacggaactggctgcaaggcgaaattctggaaaaacaactgacgtattggcaaaaccagctagccggcattactcccttgcatcgtttcccgctggataatccccggccagaaaaacaaggtttcgaaggacacgttcattcacagcgtctttcagcacatcttacccaaaaaatcagggcactgtgtaccaggcatgaagtcactttatttatgttcctggagactgcgtttgccgttttgctcagccgctacagcaatgaaaaagatatcgtggttggcatgccaatcgccggcagaagacaccgcgatattgaacccctgatcggctttttcgtcaattctctggttatccgaacggatttatccgggcagcctactttcagtgcattgctaaaacaaaacagccgcaccattctgaatgcgtatgcacatcaagatctgccctttgaaatgttagtggaaaaaatcagcccggagcgaaatttaaaccataatcctgtcttccagatcatctttgccgtacagaacaaccagcaagatactacgctggagcaggataatattgatccggatgaagaaaagacacttcttacgacccgttttgatctggaagttcatgtttatgaagacgaagagaaaggcgggctatcgattttatggatttcagataccagcctgtttaacagagataccattgacagattgctcgctaattacgaaaccctgttgactagcattgtcgaggctatgaatgtcagtccggccaataaagagccaaccattgataaacttccactattaggtgatgtggaaaaacactcattattgcatgagttgaatggatcacagactcagtaccaacgcggtctttgtttccatgagctatttgaagaacaagttgcgctcaatcccgaaaaaaccgcattggtttttggcgaaagcacgctcagttatagagaattgaatgagcaggccaaccaattggcgcattacctgattgaacagggtatccagcctgataccttggttgcaatttgtcttccgcggacgctgcaaatggctgtcacactactgggtatcctaaaagccgggggggcttatgtcccgttggatatcagctatccgaaagcgcgtcttcaatatatgctggaacacagcggggcggaatttattctgactgaaacgcatttagtcgataacctgccactcaatcaacaaaatgtcatttgtttggataccagcgaaatacaatcacatctaaaaaatttaccaacagaaaacattatcgaacgcccattcccactgactgaaaaccatttggcctatgtgatttatacctctggctccacaggcagaccaaaaggcgcaatgctggaacacaaaggctgggttaatctggcacacgcgcaggctgcgttatttggggctgattcaaatatccgcggattacaatttgcctcctggagttttgatgctgccattctggaaatgtccatgacattggcatacggggcaacactgtatcttatttctgaaacccagcgccgttcccctgagttactggatgccatcgttgaaaaatatcagattactcatgccgtattaccgccagcgttattacctcatctggatttcagcaaatggcgcagtgtttcaaccctgttgttagccggggaggctgtgccaccccatattgccgctcaatggtcacaagagcggaacttgttcaacgtttatggcccgacagaatgtacctctattatcacctgcggattattaacggcagataagcggatcacgattggcaaaccactgccgaatgccgtgatgcgcattatggactccgatggcaatttagtcccgcttggtgcggagggggaactgcatatcggcggtatccagttggcacgcggttaccggaatgcgccagaactgacagaaaagcagtttatttgtgatcccttcagccctaacccgactgacaagctttatcgtaccggcgatttggtgcgctggacccctgaaggtgaattggattttattggccgtattgattcacaggtcaaaattcgcagccatcgaatcgagctgggtgagattgagtccgttctggcgggacaggatattttaagcagtgcggcagtgatcacaaatggtgacagtgatgataagaaactgattgcttatgtttgtccaagtacggattggttggcagaaaaagccacagcttttaacgctgactatcttgaaagctggacaaaaatctttgatgaccaatatcgacagacagcgactgaaaacgttatttccgataaacatgacactgacagtgattttggcggctggataaacagttataccaatcagccgatcccgcttgagcaaatggaagagtggcgtgcgggtaccatgcaacgaattgacctcctgcatccccgtcgtttactggaaatcggttgtggtacaggtttactgttatatcgctatgcggaacagtgtgaatcggtattggcgacggatatttcggcagaagtactggcgcgacatcagcaaatcctgcaacaacgcggttggtcgcatgtccagttacgtcagggggatgcgctgaatttgggtatgatcacgcccggcatgttcgatacggttgtcattaactcggtggttcagtatttccccaatgtccagtatctggacaatgttattgctcagttactgcctgcggttgaagcaagtgggaaaattctgctgggggatatccgcaaccttgatctgctgacagcacatgtgacggcgatagaacaaagccatctgggtgaacagcgtatttccgtgggaacactggcaaaccgtattcaaagacatttacagcaagaagaagagttcctgctcagcccgacttattttgctcaactgccaacacgttacccagagatcggcagagtcgatattctggtcaaacgtggcgtaggcgataatgagatgctgcgttatcgttatgacgtcatactgcataaaaaaggtgaaaatgcacaatcttgtggtcatgctcctttcacttggcatgattttgtttcccttgaaaatttacgtgatatgttacaaagggaagagcaaacattcggtgtttcgggtatccccaatgcccgcgttaaagacgatcttgccttggctgaggggttacgccattggccggccaatcaatttatttcttcatctgaacaagcgggtaatttctcctcgcaatcagtggaacagattcaatctcttgaattactgctccaatatgctgaacagtgcggttatcagtgtggtatgacgtggtcacaacaacaacctgatctgctggatgtgattttcagccgtggaacattgcctcaaatacaggctcgttctgattacagtcaaacccatctggctaattatccgcaaatttcgtctattagtggtgaactttcagaactccttgaatctgcgctgaaaaagcaattgccggaatatatggtgcccagcctgtatatccccctggagcgtatgccactctctctgaacaataaaatcgataaaaaagcactgccggttcctaacgaggatgatttgcggcggcaggcttataccgctccaagagatgaaactgagaagaagctttgtcagttatggaaaaatttattgaaagttcatcaggttgggattcatgacaatttcttcacactgggcgggcattcacttcaggcaacacgtttgattagctcaatacgcagtgaattagacgttgaaattccattaagaactatttttgagcacccgacactggagcaattatcaaaagttgtcacagttcatcttgtcatggcaagaagaaaacatttccagacagagcaagggacaacacaaaaattattgaaaggtgatatatga

SEQ ID NO: 4 - DNA, 5997 bp, Xenorhabdus nematophilus
atgaaaaacgccgctcagattgtcaatgaagctttaagtcagggaattaccctgtttgttgctgataatcgcttacaatatgaaactcagtgcgacattataccgccggaattgcttaatgaatggaaagaacataaacatgaattgattaagtttctgagtgacgccaattcagacgaggaaacacacacctatcaattgcaagctatccaacgtgatgagcatgcaaccgattacccactctcatttgcccaacagcgtttgtggttgattgatcaggctaatggcagcagcccccagtacaacggtaccggggatttcagattaaaagaaaccgtgaatattgaggcacttgaagcggcagtgaagagcttgcttgaacgtcatgaagcattgcgcactcattttaaaatcattgataacgaaccgcggcaggtgattgcgacaacttacaatctgcccgttatttatcatgatttatcggcactgtctgaaacagacaaaaaccatcacttaaaacgactcaataaagaagaaggtgatcagatttttaacctcagtaccgatctaatgctgcgtatccgggtcgttaaattggccgaaaatgattacgttattatctatacgatgcaccatatcgcctacgatggctggtcgatcccgattttcctcagtgaatttttcaccttatatagggcttattgccacggtgaaaataatccgctacccccgctaaagatccaatatgccgactatgcccgatggcaacggaactggctgcaaggcgaaattctggaaaaacaactgacgtattggcaaaaccagttagctggcattgtccctttacaccattttccgttggataatccccggccggaaaaacagggcttcgaagggcgtgttcattcgcaatgtttttcagagcatttcactcaaaaaatcagggcactgtgcgccaaacatgaagtcactttatttatgtttctggaaacggcgtttgccgttttgctcagccgctacagcaatgaaaaagatatcgtgattggcatgccaatcgctggcagaagacaccgtgatattgagtccctgatcggctttttcgtcaattctctggttatccgaacggatctatccgggcagcctactttcagtgaattattaaagcaaaacagccgcaccattctggatgcttatgagtatcaagatctgccctttgagatgttggtggaaaaactcagcccggagcgaaatttaaatcataatcctgtcttccagatcatgtttgccgtacagaacaatcaacaagattttatttgggagcaggaacatattgttcaagatgaagaaaaaccgcttctcacgacccgttttgatttggaagtccatgtttatgaacaggagaatgagttatgggttgtatggattgcagataccagtctgtttaacagcgataccattgacagattactcgctaattacgaaaccctgctgaccagcattgttgaggcgatgagcgaccattcggttaataatgagccatccgtccatgatctcccgtttctggctgatgcagaaaaacatatgctactgcatgagttgaatgggccacaggcccagtatcaacgcgatctgtgtttccatgagttgtttgaagagcaagttgcactttatcctgaaaaaaacgcgttgatttttggcgaaagcacgctcagttatagagaattgaatgagcaggctaaccaattggcgcactatcttattgaactgggtattcagcccgataccttgattgcgatttgccttcctcggtcattacaggcagtggttgtactgttgggtatccttaaagctggcggggcttatgtgccgctggatgccagttatccgaaagcgcgtcttcaatatatgctggagcacagtggggcggaatttattctgactgaaacatctttggtcgaaaaattacctgtcagccaacaaaaagttatttgtctggacagtgacgaaatacagtcacatgtgcagaacatgctggcagacaatattgttgatcgacttttgcccctggcagaaaatcatctggcctatgttatctacacctccggttcgacaggcaggccaaagggcgcgatgatggaacacaagggttgggtcaatctggctcatgcgcaaaccacgttgtttgggattgatacagatagtcgtgtattacaatttgcttcctggagttttgatgctgccattctggaaatgtccatgacattggcatacggggcaacactgtatcttatttctgaaacccagcgacgttcccctgagttactggatgcaatcattgaaaaatctcagattacccatgccgtattaccaccagcattattacctcatttgggtttcagtaagtggcgcagcgtttcaactctgctattagccggggaggccgtaccaccgcatattgctgcacagtggtcacaagatcggaaattgttcaatgtctatggtccgacagagtgtacctccattatcacctgcggattattaacagcagataagcggatcacgattggcaaaccactgccgaatgccgtgatgcgcattatggactccgatggcaatttagtcccatttggtgcagaaggggagctgcatatcggcggtattcaactggcccgcggttaccggaatgcgccggaactgacagaaaagcagtttatccgtgatcccttcagtactaacccggctgacaagctttatcgtaccggtgatttagtccgctggaccctcgatggtgaattggaatttattggccgcattgattcacaggtcaaaattcgcagccatcgaatcgagctgggtgagattgagtccgttctggccggacaggacattttaagcagtgcagtagtgatcacaaatggtgacagtgatgataaaaaactgattgcttatgtttgccccagtgcagagtggctggctagaaaagctaccgaatttaatgccgattatgtgcaaagctggacagaaatttttgatgagcaataccgacagaaaagtgtcaaaaataccctcgctgatcagcatgatgcggatagtgattttggcggttggataaacagttataccgatcaacccatcccgcttgaacagatggaagagtggcggacgggcaccatgcaacgaattaatgccctgcatccccgccatttactggaaattggctgtggttctggtctcctgttatatcgctatgcggaacagtgtgaatcagtcctggccaccgatatctcagccgaagtgctggcgcggcatcagcacagcctgcaacaacgcggttggtcacatgtccagttgcgtcagggagatgcccttaattcgggggtattggcaccggacacttttgataccgttatcattaactcggtggtgcaatacttccccaatgttcagtatctggacaatgtccttgcccagttattgccggcggttgccgcagggggaaaaattttgctgggggatatccgcaacttagatctgctgacaacacatgtgacggcgatagaacagagccatctgggtgaacagcgcatttccgtgagcacgatggccaaccgtattcagcggcgcttgcagcaagaagaagagttcctgatcagcccgacctatttcgctcaattacccgcccgttatccggagattagccgggtggatattctggttaaacggggcgtcggtgacaatgaaatgctgcgctatcgctatgaagtcatcttgcataaacgggataaccatgctgcatcatgtcacgatcggcctgtcacctggtttgatttcaatacgattgacgcggtgagccgtctgctgcaagcaggaacttacaagacctttggtatttcaggtatgcccaacgcccgtatcaaagaggatgttgaattggctgaaggactgcgtcattggccggccaatcaaatactatcttcactgaaaaataccggctgtttgacctcggaagcgatggagcaaattttaactttcgaatccttgcttcagtatgcggagcagtgcggttatcaatgcggcgtgacgtggtcacaaaaacaatcggacttgttggatgtgattttcggtcgtgatgagttgcctcctgtccaggcccgtcatcaatacagccaaacccatttagccaattatccgcaaatctctgctattggaggggaactttcagagcaactggaaatggccctgaaaaaacagttgcctgactatatggtgcccagcctttatatcccactggagcgtatgccactctcgctgaacaataaaattgataaaaaagcgctgccggttccagatgacagtgatgtacaccatcaaaattatgtcgcccccagagatgaaattgaggaaaaactctgtcagttgtggcaaaagcatctgaaaatcaatcaagtgggtatttatgataatttcttctcactcggtggccattcgctcttggcagtgaaaataacagcctccataaaaaatgatttaagtaacaatttcagtatgaatcaaatgttttcgaatccgactattgcacagattgccagtgtgattcgtagcgctcaagaccatgacatatctacactattgccaacacaacgtgtatccgacggaaaaataccaatttcttattaccaaaagatgttctggtctctgatccaagaaagagtattggacgatgcctttaatgttcccattaccttgttgatggagggcgaattaaaccaaagtgcattggaaaaaagtttgactgttattttagaacgacatgaaagtttacgattaagattttatgaagaaaatggacagtttttcatgaaaccgaataacagcatgaaaatcaatttacaaattatcgattcgttgccaaaaggaatggagaaagaacaaattaccattgaaattaacaaactctgggaagagaaattaagagtaccttttgatatttacaatggcccattattcaagacatttttgctgcctgtatcagaaacacgccatgtcttattagttaatattcatcatattatctgtgatggatggtctatcaatatttttatgaatgaatttaagaaactatactcggcttactcacaggaacaagatattattcttcccatagttgcaatgcaatattctgactgtgtttatgatttgcaagaacgacataagactgaaaaatatcaaaggcagctaaaattctggctgcaacaatttgctgatattgatatgaatgaacgttgttttccagtcgctttcagcaaacatctgactactgagtatcaatttaaagcgattaatattaatactccaaaatcattggataaggctgctgtacaatattgcgaaatccataagattacaccttatatgttatatatggctttgttccacgcttgtctttttatacattctggtgaatcacaacatatcgccacatcaccacaagctgatcgttctcatattgacagccatcaaacaataggactgtttttgagtgacctgatagttaaatcaactatcagtaaagagatgagcctttatgaaatcattcagcaagtcagtgattttatttataaagcgctggaaaattcagatatccatatggctgcggtgattgataacgtaggggaaaaggtgagagataatatatttaatattgtctttaactatatggatgctcagaatctctatgattatgcagataattctgatagtggaaaaatatccctgccaagcctcgatatagaattgataaatattgacccgactccttttcaatcactggggataaacttcatcaacataccacaaaatatcatgtgcgagttaagctataacccagagctgtatactgaagaaactgccaataatcttgcaagatattatggacgactgctggaagccattgtctctggtcaagataaagaaaatatcagttacttttatgactaa

SEQ ID NO: 5 - DNA, 17042 bp, Xenorhabdus nematophilus
ggaaccctaaagggagcccccgatttagagcttgacggggaaagccggcgaacgtggcgagaaaggaagcgaagaaagcgaaaggagcgggcgctagggcgctggcaagtgtagcggtcactctgagcgtatccaccacacccgtcgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagaactagtggatcccccgggctgcaggaattcgcgtaaagtgaagcttgaatcagacgttctcaatgctcgcttggcggccgaaaaaattgatgtttcattgcctggtcgtcggatagaaaatggcggattacatcctgttacccgcacgattgaacgtattgaaactttctttggcgagttgggcttctctgttgagtcaggtccagaaattgaagatgactatcataactttgatgccctgaatatctctgctcatcatcctgcccgtgctgatcacgacactttttggttcgatgcaacgcgcttgttgcgtacccaaacctcaggggtacagatccgtaccatgaaaaatagtcagccacccattcgtatcattgcgccagggcgtgtataccgcaatgactatgaccagacacatactccaatgttccatcaaatagaagggctgattgttgataaaaatatcagctttactaatttgaaaggtacactgcatgactttttgaataatttctttgaagaagatatgcaagtccgtttccgtccttcttatttcccatttacagaaccttcggcagaagttgatgtgatgggtaaaaacggtaagtggctggaagttttgggctgcggtatggtacatccgaacgttttgcgcaatgttggtattgatcctgaaatctattctggttttgcctttggcatgggaatggagcgtctgaccatgcttcgttatggggtgacagacttgcgtgcattcttcgaaaatgatttgcgtttccttaaacaatttaaataaggcgggtatatctcatgaaattcagcgaactctggttacgcgagtgggcaaacccagccattagtcgagaagcattgtttgatcaaatcaccatggctggtttggaagttgatggtgttgaaaaagtggcaggccaattccacggtgtatttgttggtgaagttgttgaatgtggccaacatccaaacgcagataaattacgtgtgacaaaagtcaatgtccctggcgaacgcttgttggatattgtgtgtggcgcaccaaattgtcgccaaggacttcatgttgccgtagctacagtaggagctgtactacccggtgattttaaaataaaggctgccaaattacgtggggagccatcagaaaggatgctgtgatcgatttctgaacttgacattgcagatgatcacgatggcattattgagttgccagcagatgctcccattgggacggctctgcgcgaatatatgaaactggatgacagcgcgattgaaatcagcattacaccaaatcgtgctgattgcttgagcattatgggcgttgcccgtgaagtcgcagttatccaataagctatcgatggttgaaccaaaaattgaggtagttaaacccactattgatgcaacattccctattcgtgttgatgcgtctgaagcatgtcctcgctatttaggacgtgttatcaagaatgtgaatgttaaagcggttacaccattatggatgcgtgaaaaactgcgccgtggtggtatccgctctattgatcccgttgtcgatattactaactatgttttgcttgagctagggcaacccctgcatgcgttcgatttggatcgtttacatggttcaattattgttcgtctggctgagcaaggtgaaaagctcacactattagatggtagtgaagtcgaactttctgctgaaacactcgttatttctgatgaaaagcaatcaattgctatggcagggatttttggtggagaatattctggagttaatgcggaaactcagaatattttgttggagtgtgcatttttcgcacctctggcgattacaggtcgcgcgcgtcgctatggcctgcatacagatgcttctcatcgttttgaacgtggtgtggatccacagcttcagcataaagcgatggaatatgcgactcagttgttagttgaaatttgtggcggtgaagcgggagaagtcattgatgtgactaacgaatctcatttgccaaaaccagcaatgattactctgaaccgtcagaaattagatcgtttgattggccatcatattactgatgaacaagtcaccgatattttgacccgcttaggctgtcaggtaaccgtacaaaaagacaactggcaggttgtcgcgccgagctggcgttttgacatgcagattgaagaagatctggttgaagaagttgctcgtatttacggttataacaacattcctgatgtgccagtgcgtgctgatctcgtgatgacatcacatcgtgaagctaatttaccattaaaacgtgtcaaaacaatgttggtggataaaggttatcaagaagcgattacctatagctttgttgatcctaagattcagcaatatctgcatccgcaacaagaagcattgattctgccaaacccaatttcggcggatatgtctgcaatgcgtttgtctttgttgactggattattgactacggttgtatataaccaaaatcgtcagcaaaaccgcgtccgtttatttgaaacggggttgcgttttgttccgtatgagaatgcggaatacggtattcgtcaagaattaatgctggcgggtgttattgcagggaaccgttttgatgaacattggtcgattgaaaaacaagcagttgatttctttgatttgaagggtgatttagaatccgttttagaactgacgggtaaactctccaagatttccttcagagcagaagaacatcctgctttacatcctggacagagtgccggggtttatctggaaaatagttacattggtctatattggcgttatccatccagaactggagcgtaaacttgacttaaacgggcgtaccgtggtattcgagcttattatggtctggtctggctagatcgcgtgattcctgagatgaaggaagtttctcgcttcccatctaaccgccgtgatattgctatagttgtgcctgaaaatgtagccgcagaagatattcttgctgaatgtaaaaaagttggcgtaaatcatatagttggcataaacttatttgacgtgtattgtggtaaaggtgtcgcagaaggctataaaagccttgctattagcttaatcttgcaggataccacgcgtactctggaagaagaagaaattgctgcgaccgtcaacaagtgcgttgctgcattagaacagcgattccaagcatccttgagggactgaacttatggcgcttactaaagctgaaatgtcagaaaatctgtttgagaaactgggtattagcaaacgtgacgctaaagatctagtggaaatattctttgaagaagtgcgtcgttctttggagaatggggagcaggttaagttatctggatttggcaactttgatttgcgagacaaaaatcaacgtccgggccgcaatccaaaaaccggcgaagatattccaatcccagctcgccgtgttgttacttttgcgcctaggcaaaagtctgaaaaatcgggtcagcagaaagctttacccatagaacatgaacatgaaaaaggccgcataaatgcggcctttttgtcatctgtcatattctgttgcatgcaatgggtggctttccctttttcttaaatggtttcatatcaccatgcactctgtttatgacctgatcattttgcagagaaggcgtaactataaaatagtcgctatcttatctgttttactctctattctatttattttgtcattatgtgcatgagaaatttggttttggcctgatgaatggctgtcagattcagcccaattatttatttggcaattgaggttacccagagcgattgccgtgatgatagatggcgcaagtttggccgtatcaggagcaatcatgcaagccttgtttgaaaatccattggcagaaccgggattgcttggaataagtaatggtgccggggttgtcatcgtatttttgattcttgctttccacggtatggcaccattatggttattaagcatacggagcggtatttggcgcactttgtatgactgccattctgttgggattttcccgaaggcatcaagtcactaatactcgtttgttattgattggtgttgcctttggcgtgatatttggcgccatgatgacatggatggtttatttcagtaccagtctggatctccggcagttaatgtattggatgatggggagtttcagtggcattgattggcgtcagaaatggttagtcattgcattgctgcctgccattttatggttaagtcgtaaggggagtattttgaattatttgtcacttggagaattacaggccaggcaactgggaatctctcatcataagtggcgtaatttatttgttgcagtcgttgggctgttagtgggtttgagtgttgcccttgctggagcgataagttttatcggtttggtcattccccatattttaagattgattggcgtgacagatcacaaaattttacttccggccagtgcattggcgggaggatgtggtttattgttgcgctgatttattctctcgtctgtcattatctcacgcagaagttccgataggtgttgttaccgtaacgttaggagcaccaatctttatatggcttttattaaaaacgcggcattattaaaaacacatcaaatatatctctgccggttaataaaaatccgtctgtgagataaggataattatatggcagaccagccgattgcagttttaaataatgtggctgttgataacagactttactcgctatctgcaactattatggcgggggaagaaattcatctggttgggctgaatggttcaggcaagagtactttactttcagcaattgcaggggttttaccctacaaaggcagcatttatctgaatggcagaaatttaggtgattatagtcaaaatggactggcaaaatatcgtgcttggttttctcaatcagcttctacacccattatgcctgtttttcagtatttggatatgtttcgtcccgaatcggttccgttggctcaaagtgaaaaagttctgtacagactttgccagcaattgaaactgcttccgttacttccttcacacaccgggcagttatcaggtggagagtggcaacgggtcagactggccgggacttttctgcaagtatggccggagttgaaccccaatggaaaattacttttgcttgatgaaccaacgaataatcttgatattacccagcaagccattttggatcagttggtcagagaattttgttcattgggcggttcggtgctattgagtagccatgatcttaatcacacttataagcaggctaccggagtatggttgctttctcacgggaagttattggcatcaggtatgccaaaaaatgttatgacagaacgaaatttatcagaaacttttgagataaatattagacatatcagaaatacaagctataagttatgggtggtcagccatacgtaacttatagcgattgaaataaccataattgattgctttttatctactttaaatgttttgattgaccagaatttatctgcctgttgatgtttcgttaaacaaacatcatctaggattgtccaagtataaaacaaattaggcgatgtagggcataaggcgattatggatagttttttgccattttctcgtcctgcgattggcagtgaagagatagaagcgattgagaaagttttacattctggttggattacaacagggccacaaaaccatcaattagagcaagatttttctcgtatgtttgggtgtaaacatgcggttgctatatgctccgcgactgcggggatgcatttggtcttattggcattaggcataggttctggtgatgaagttatcacaccttcccaaacttggatttccactatcaatatgatttgtctgctgggggctgaacctgtcatggttgatgttgaccgtgatatgttgatggtcagtgcagatacaattaaaaaagccattacaccaaaaaccaaagccattattcctgtgcattatgcgggggcaccttgtgatcttgatgcgctacgtgctgtcgcagaagaagctaatattcccttaattgaagatgccgcccatgctgtcggtacccgttataaaaatgaatgggtgggagagcgggggacggcgattttctctttccatgcgattaaaaacatgacttgtgcggaaggtggcatgttcgttacagataatgatgaactggcccatcggatccgttgtttaaaatttcatggattatctgttgatgcttttgaccgaaatattcagggtaggaagccacaggcagaagttattgagccgggttttaaatataatttatccgatattcatgcagcgatggccgttgtacagttaggcaaactggcactgatgaacgcacgccgtcgtgaaattgtggctcgttattctgctgcccttatcgattcacctttgcaaatgttgagcgttcctgaatatgaacatttgcatgctcaccatctttttatggtacgtgttgacaaaaatgtctgtggtattgatcgcgatactttcatggcacgtctgaaagataacaatattggtacaggtttacatttcagagcggcacatacgcagaaatattatcgtgaacgttacccacagctttccttgccgatatctgaatggaactcatcagcactttgttcattgccgcttttcccggatatgagcaatggtgatgtcgatcgtgttgtgaatgtgataactgaaattctttcggagcataaataagtgacatttgagaagatcaaaaaagtttctgttgtgattcctgtttacaacgaagaagaaagtttgcctcaattattggcaagaacggttacggtttgtcaaaaacttgaacaagaatatgaaattatcttggtggatgacggtagccgtgacagttctgccgatattttgacccgggcggctgaaattccagaaaatcatgtgattgccatttttcttaatcgcaactacgggcaacattcggcaattatggcgggatttcatcaggctgatggtgatctggtgattaccttggatgctgatttgcagaatccgccagaagagatcccccgtttagttgacgcggcaaacgaaggatatgacgttgtaggaacgcgccgggccaaccgtcaggattcctggttccgtaaaagtgcttccagattaatcaatgcgatgataaccagagtcaccgggcgttctatgggggattacggctgcatgctgagagcctatcgccgccatattatccgtgcgatgctgcaatgtcatgaacgcagtacatttattcctattctggctaacacttttgcacgtaaaacaatagaaattgatgtgactcatgcagaacgcgagtttggggattcgaagtacagtttcatgaaactgataaacctcatgtatgaccttctgacgtgcttaactactgcaccgttgcggttattgagtgtagtcggaagtattattgccgtaacgggctttctgttggcggtattgttgattactttgcgccttatctttggctcaatgtgggccgctgaaggtgtgtttactctgtttgctatcctcttcatgtttattggtgcacagttcgtcgcaatgggattacttggtgaatacattggccggatctataacgatgttcgtgcccgcccgcgttattttatccaaaaagtggtcggagttaaaaagaccagcgaaaatgacaatcaggaaaaaagctaatgaaagcagttatctttgcctatcatgatattggttgtgttggacttagtgcgctagcgaaagcaggctttgatattcaggctgtatttacacatacggacgatccgaatgaaaatcactttttttcatctgttgcacgtactggtgcagatcttgggttacctgtatttgcgcccgaaaatgtgaatcatccgctgtgggttgagcgcattcgggagatgaaaccggatgtcattttctctttttattaccgtaacatgcttagtcaagaaattttgtcattggctgagaaaggcgcgtttaatctgcatggttccttattgccgaaataccgtggtcgcgccccggttaactgggcagtacttcatggagagaaagaaacaggggtaaccctgcataaaatgctggctaaacccgatgccggcgacatcattgcacagaaggccgtccagattggagaaacggatacttcgttgagtgtacatgcaaacatacgtgaggcagccgttgaattactggacagcattctgccacaaataaaatctggtcattatccttcgattccacaaaacgaaaatctggcgacttattttggccgtcgaacagctgaagatggtgagataatctggcgtaaaccagctaaagaaattaacaatttggttagggcagtgacagaaccttatccgggggcgtttactttcttgggtgagcgtaaaattataatttggcgcgcacggccactcgatcaagcccatgataaacgccccggaacggttatctctacggaaccatttgttattgcttgtggtgatggcacgttagaaatcatcagtgggcagggtgaatcaggtctttatgtgcaaggtaatcggttggcatttgaaatgggcattgtagcaggtgttcatgttggcgctaaaacaacaacacaaattagtcgccgtaaacgtgttttgattttaggtgttaatggattcattggtaaccatctgactgaacgtctgttagaggatggtaactatgatatctatgggatggatattggttcttctgctattgaacgattcatcggcaatccgcattttcatttcattgagggggatgtcagtattcatactgagtggattgaatatcacatcaagaaatgtgatgtcattcttcctctggtggcaattgccacgccgattgaatatacccgcaatccgttacgtgtttttgaactggattttgaagagaacctgaaaatagttcgttattgcgtgaaatacaataaacggattattttcccgtcaacatctgaagtgtatggcatgtgtgatgacaaggaatttgatgaagatacttctcgtctgatagttgggcctattaacaaacagcgctggatttactcagtttctaagcaattacttgatcgcgttatttgggcatacggtgcaaaagaaggtttaaaattcacgttattccgtccgttcaactggatgggaccaagattggataatttggactcagcccgtataggaagttcaagggcgataacccaactgattttgaacttggtagaaggttccccgattaagctggtggatggcggagagcaaaaacgttgtttcaccgatatccatgatggcattgaagccctgttcaggatcatcgaaaaccgggatggcttgtgtgacagccaaattatcaatattggtaatccgacaaacgaagccagtattcgccagttggctgaaatgttactggagagttttgaaaaacatgagttgcgggggtatttcccattattctccgggttcaagaagattgaaagcagtagctattacggccagggctatcaggatgtcgaacaccgtaaacccagtatcaagaatgcagagcgtcttcttggctggaaaccgacaattgatatgaaacaaaccattgatgaaacattggatttctttctgcgcggtgctgttgaagagttaaaagggaaaaatcaggaagaaagcgatgaagcaagttggcttgagaattgatgtagatacttatccgggcacaaaggaaggtgtgccacggttacttgagatcttgcagaagcatcatattcaagccagcttttttttcagtgttgggccggataatatggggcgccatttatggcgccttctgcggcctaaatttttctggaaaatgttgagatcaaatgcagcctctctttatggattggatattttgctggccggcacagcatggccgggaaagaaaatcgcaaaagatttgggttatttgatgaaacagacaatggatgcaggccatgaagttggtttacatgcgtgggatcatcaaggttggcaggcgaaagttgggcgttggtcggaaactgagttggcagaacaaaaaaaattgggggttcatgctttaaaaaaagcagtagggcaaccgattacatgttcagcggtggcaggttggcgggccaaatgaacgagttttggcggtcaaacaccattttggttttgattacaacagtgattgcccgtggaacgcacccattcaggcctctttttacctgatgggaatttaggtacggtaccaaaattcctggttaccttgcctacttatgatgaagttgttggaacacagatcggtgatgaagatttcaacagttttattctggatgctattaaaacgtgatagtggcgtgcctgtttataccattcataccgaagtggaaggtatgtctaaatcttcgcagtttgaaacattactgaacatgcttcatcagcaaaatattcaattctgtaagttgagtcatttattgcctgaaaatagaaatacgcttcctgttgggaaaattatacgttcgaattttcctggtcgtgaaggctggttaggttgccaacaaaaggtataacagaaatatgttgaatactcgggcgagtaaagcaggagccgttctgatggctcttttttttatcctgacttacctattaccactggatggtcgtctattatggcagccggatgaaacacggtatgctgaaattagccgggaaatgctgcaacacgggaactggattgtaccttattttttagatattcgttattttgaaaaacctgttgctggatattggattaacaatatcagccagtggatttttggtcataataattttgccgttcgttttggttctgtgtttagtatcttagccagtgcttttctgctttatcggttggcgatgatgatgtggcaatgccgtcagactgcgtttgtttccagtttgatttacatttccatgtttattgttttttcaataggaacatatagtgtccttgatcctatgttctctctgtggatcacggcgggaatagtgagttgctattgggcacttaaggctggcagtatccgggagcggctcctggcgtggtctgtgctggggttgacttgtggcatggcatttatgacaaaaggcttcttagcattggcgttgccggtgattgccatgatacctgtcacgatataccagaacgttttcttgaaatgatttgttttgggcctgttgccgtcattttcgcaattttgatcagcttaccgtgggtgattaccgttgctttgagtgaatcccattattggcattatttcttctgggtagaacatatccagcgttttgcttctgataaagcgcaacatattgcccccatctggtattacttgccaatactggttttgggcgtgatcccgtggttaggtctgttacccggtgcgttgatcaagagctggaaagagagaaacagtcatccagagatgtttttcttgttttgtttggtttattgcgccatttatatttttcagtatcgcaaaaggtaaattaccgacttacgttttaccctttatagggcctttggcgatcatgatggcaaaatatagtgttgattgtgtgagaaatggcaacatgaaagcactgaaaatcaacggactggtgaatgtattcattggattgatagcaattttggtgctctttggtatggaaacactggttaatcctccactttatcagcccaatgagtggcttaaatggattatggggattattgcctttggattttggggcgttgttggttatctgtgtttcacacatgatggcaaacattggttatgggctgctgcttgttcaattgtgcttagtttatcgataggtagcgcactgccttccaaaacaattaattccaaacttcctcagttttttattaggcaaaatgagaaagcattggcagacagtaaatatattctgtctcagtcggttggtgtcggagcggctattgcatgggaattgaaacgcagcgatatttacatgtttgatcggccgggagaattggaatacgggatttcttacccagatagccaccaccgatatgtgacaggaaaagaattttcctgagtggttagcaaaaagcaccgtaaaacagggacaagtatcgattgtgttcctgctatcttccaaaaggttcaccttcccgccattaccggagcccaaaatcctggggccccagcaaatcacccgttttggtccccgggaatttaaaaaaaaaaccccccctggaaccgtaaatatcgcgtttggttaattctgggggaattttattgacctgtgcagggcaattatgtcaaaaacaggccgttatctgttggcaaaaaaaagatatccctcataaagggatatctatggtgatgtggctttttggtgctgttcttctgttgggaatgggaatgatattctggctgagattattacaatttttgccgctgggtattgcatatcctatgttaacgtataaattttgttgttgtgactttaatgggtcagtttatatatcgggaaaaagtggggccaaaacactgggtaggtattttggcgattatgtttgggatcttattgatgagtttagagcaatgaaaggttatctttgggcagtggctagcgtttttctgatcactatagcccaattattattgaaatggggtgtcgctcgtttgccggagctttcgttgtcgtcaatgcattggctgaatagtgcctggctttggcaaaatcataacgctttactgatgattatggcaggcttggcgggttatacactatctatgttatgttggttttttaccttaaaatatctgccattgaacaaagcataccctattattagtctgagttatgtgcttgtttatttgatggcggctttacttccctggtttaatgaaacgccttcactattaaaaacagcgggggttatgtctatcttatggggagtctggttaatcagccgaccagaaaaaagcagcctggttgatcgtcatttataattgattagttggcttgatataaaagggacatccgaaaacgtcctgtttttttatctcatgccagataaagtttgtgcgtttttgtatgatcaccgggggaaagcaaatgataataagagcaggttatttagcttattatttagttttttcctcattggttgtagtaatccggatccgccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaaatctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggatccggtcagcaaagaagagctgatgccgggagatttagtgtttttcaagacagggagtggtggaaatggcctgcatgtcggtatttatgatactaacaatgagtttattcacgcatcgaccagtaaaggggtcatgcgttcttccctcaacaacgtgtattggcgtcgggttttctggcaagcccgcaggatgtaatcccacgtaccgcccgacttacagacgtcgctatccggaattattggctactcggacgaacgcattgccaccgcagaacaaatgattttggggttgcaggagtatgttagggcggaatgtttgagataaaagcccctgcaataaggggctaaatattagctaactgaaagtgatagtttttttcctatagcggcaatcgcccggctaatcgtatcaattttagttgcatggtgtggtgcgagtattctttggatttcaggggggcggatacccgttaagcgtgataattcagccttattgatgttagcctttagcaactcattattgagtaagattttggcatacacgctatcaggtaaatatacccactgttcaccttcttgaatctcaggatcaggcagaggaaagttattatccatatcaaagtatatttcgacacaggaaagtaaaacatcctcggccatttcctattgcttcaggtatatctgcaccacaagtgatagcttcggggatatttctgaatgtgactgtatatccgttatcttctttaataaattttgcagaatagtacatattggcctctctaacttatttattgatgcagtgacagcccctgataatgagggctggaatttatttcaaacctaactgtttctttactccctcaaccaagcccgttgttaattcttgactgggatgtctggggaggtggcttattttgcctttgtagtttcatggtaaaagtgccaaaagtgttaggcaaggcagctgaaaaatttcttgcagttgatgttttcattaacggcacgggtggacgagttgagcgagatatttaccgcttctttaagcgtgaatcactgtctgatggcgatgagctacagctacgagttacagactcaggtgttcgaccacatgacggggcttgagcctaagtctggatttggatttaccatccagcaactgcaacatatgctggcagtggctaaaaaatcctctgatgaggattcatctgatgtcggacgaaggactaagaaaaagacaggatgatttagtgatcctgaatagaactgaaaagttaattggcgatattagtcaaatggcacttggtcttgttggcggcggtaaaaaggcgatcactcattagatagacatcgccaccccagagcaaatgattcggttgcagaaaccatcctggctcctgcttgattgttgttataaacagtatttagtcggtttagctggatttaacacaagacaggcagccatacactctgcatatgctatgggtgatatacctaaaggtaagcagagataagcataagtctgcattgaagctaaaaaagcagaacccgccaacaggcaaagaagaatatattttttcattttattctccgtatgttacctacagtaccacagaacctaataaccccatttcaactcaattaaaagcgaataatactgaacaattgattacccactgaaactaaacaaactatttctttacctattcccttgagtggttaaagcaataacccctgccaccgtataggacgggtcattatcagcaatatccgctgtaggcagaagaagcggtgtgacagccggagagacggcctgcattgccatcatgattcatggagtcgtgattacaatgcagctaaagacccatgtccgatgatcggacattgggcagattctttgtaagagcattattgacgcctttttcgtatattttggtgaaacagttaaagaagaaataatttctccgcaggctctgcctgatttccccggttttagtgatatttttgctaaacaaagtagttgggaatggaatttcggtcaggcatctgcatttagccatacattggagatgtggtccggttggggaggtgttgagctgtatattgatgttgagcacggtgtaatcagccgttgccaaatttttaccgatagtttggaacctgccccgtttgaactattggcaaatttgaccaaagaattgcagcaattccacgactggattacctgttcattgaagtgatcagacaatttgatacagcataaaaaaatggtcaacgacagggaagttgaccataaaaggtgataattattataaatctttattccccacactgtaggaaagttttgaattcgatatcaagcttatcgatacgatacgacctcgaagggggacccggtaccagcttttgttgccattt

SEQ ID NO: 6 - DNA, 741 bp, Xenorhabdus nematophilus
atgagcagtaatatcgcgttgttaattctggtgagtttattgacctgtgcagggcaattatgtcaaaaacaggccgttatctgttggcaaaaaaaagatatccctcataaagggatatctatggtgatgtggctttttggtgcctgttccttcctagttgggaatgggaatgatattctggctgagattattacaatttttgccgctgggtattgcatatcctatgttaagtataaattttgttgttgtgactttaatgggtcagtttatatatcgggaaaaagtggggccaaaacactgggtaggtattttggcgattatgtttgggatcttattgatgagtttagagcaatgaaaggttatctttgggcagtggctagcgtttttctgatcactatagcccaattattattgaaatggggtgtcgctcgtttgccggagctttcgttgtcgtcaatacattggctgaatagtgcctggctttggcaaaatcataacgctttactgatgattatggcaggcttggcgggttatacactatctatgttatgttggttttttaccttaaaatatctgccattgaacaaagcataccctattattagtctgagttatgtgcttgtttatttgatggcggctttacttccctggtttaatgaaacgccttcactattaaaaacagcgggggttatgtctatcttatggggagtctggttaatcagccgaccagaaaaaagcagc

SEQ ID NO: 7 - DNA, 504 bp, Xenorhabdus nematophilus
atgatcaccgggggaaagcaaatgataataagagcaggttatttatgcttattatttagttttttcctcattggttgtagtaatcctgtctctaaatccaactcttcgccactgagatccgccctttctgatccgatagtggtaatagctcagctaaaagatcagcttgaaaaatggcatagcgcaccataccgttatggtggaatggacaaacagggtattgactgttccggctttgtctaccgcacgttttatgatcgcttcaatattcggctcccgcggaccactgacgaacaaactaaactgggcacacgggtcagcaaagaagagctgatgccgggagatttagtgtttttcaagacagggagtggtggaaatggcctgcatgtcggtatttatgatactaacaatgagtttattcacgcatcgaccagtaaaggggtcatgcgttcttccctcaacaacgtgtattggcgtcgggttttctggcaagcccgcaggatg

SEQ ID NO: 8 - DNA, 3361 bp, Xenorhabdus nematophilus
ggatatattcaacaaggacgcattagcatcgatggtaaaaccgcaacactgggtgatcgcatcgaagttaagccttcaataaaaatccgtctggatgggcggatcttaaatatcaaagaaccgcaaaaaacagtttgccgcgttctggcttattacaaaccggaaggtgaattgtgtactcggcatgatccagaaggtcgtccgaccgtgtttgatcgtctgccaagaatgcatggccacgcgtcgactagtacagggcgcttggatatcaatacctgcgggttgttgctgtttacaacagatggcgagctggcaaaccgcctgatgcacccaagccaggaagttgagcgtgaatacgctgtgcgtgtttttggtgaagtggacactgccaaactccgccaactgactcaaggtgttcaactggaagatggtcccgcttctttcaaaacgatcacctttaaaggtggggaaggtatcaaccagtggtacaacgtcactctgactgaaggccgtaaccgcgaagtcagacggttgtgggaatcggttggtgtgcattggccagcgcgtcgactagtacgcgttatggtgatatcaatctgccaaaaggtctgccgcgtggcggttggactgaattggggctggaacagactaactatttgcgtcaattggttggtctgggcgacgaaatggtgaccaaagtggccgttgaacgtgatcagcgtcgtatcaaagcgaaccagattcgtcgtgcagtgaaacgacatgcgaaggtggcatctcgtccggcaggcaaacgtcctgcaagtagttctcgccaaaatgcagggagaaaaacgacacctaaaggtcgttaatatcttcaaaccgctgtttcagtttatggcaacatattattgaaacagcggatttactttaccaatcaacgcctttttgtgcttttactccggcatcaaaagcatgttttaccggacgcatttcggtaatggtatcggcatattccagtaattctcggtgacatcctcgtccagtgatgatcacactctgatatgacgggcgctgtttgagtgccgtcagaacctcttcaagctccagataaccgtagctgaccatataagtcagttcatccagaacaacaagatttaatgaaggatcagacaacattgccaaggcatgtttccatgtggtttggccaccgcggtgtctgtttcacggttttgcgtctcccacgtaaaaccggttgccattatatgaaattcaacgccttgctgtgacagtaaatttctttcgccactttcccatgttcctttgataaattggataacgccggcgcgctgaccatgcccgattgctctggcaacagtaccgaatgccgccgtggtttttccttttccgttgccggtgaatacgataacaataccccgttcttgttgcgcagcttcaatacgggcatcgactttctctttcagacgttgctgtcttttttggtactggctgttatttctggtcattattcagcaggccccgctttgcgaccgggttgagcatcaaaactgataccggaatcatttcggctgtcatcacccatgatataaagatagactggcataatctccgcaggtgtttttaatttggctgagttctcatcgggaaacgcagtggcacgcatgttggtacgggttccccccggattgatgcaattaatgcgtagggcagtgtcttgatattcttgtgttaaaacctgcataaagccttctgtggcaaatttggaaacagaatatgctccccaaccactccggccttcacgtcctacgctggagctggtaaagactaaagaaccgctggcggattttaataacaggggaatcaatgcctgagtcagcataaaagtggcattgacattcacttgcatcacatcatgccaaagctgcacaggttgttccacgaccggtgcaacaacaccaagaagccctgcattgtgcaaaacgccatccagacaggaatagcgctgtgccagttctttggcaaaagtctggcactcttctgcggtgacagtcaggagatccatcgtatacacgctggcgggtaaacccccttcgttttcaatttgttgtttgactgcttccagttttgcggcagtacgccccagcaaaatgacctgcgccccgtggcgggcataagtcagtgcgacttcacgcccaatgccatcaccggcaccggtgactaaaataatcttttgttgcagcaaatcatgacggggttggtaatcaaacatcgggcagtcttcctgtattgcttgtaaatctgttagttgccatctcagagtggcataggggtttatcatcagaacaaaacttaacgctgtcgtcaaatgtgaccaaaataatggagatagctttaatagttataaagttaatgggaataccgttggctaagtaaactgttagagcacggtttcctgtttttagttagaatgaaagacattgtttatcataaattaactaaggtggatcggtctctgtgaaaaaacagggataaaacaagttatacaggaaacgatgtcaggaaaattaaccgaaatcatgtccggcctgcatgtgccggtaacggcaccgctgtcctttcgccgcaaggcgcaatatcaggtgaaatgttaccggcggggtcagcgtaattacgtccggctgaaggaaaaaggcaccgggtatctgaaaattaacgtggggctgttctggcggctgttaagccgtgacagcgggcaatcgtgggagctgatgcaccacgaacgctacaacaatgaaattcgtaaatcgtaaatgtgactgaagtcgtgtcattctgcgccttgtgcaacgccacagggtgcagccctgacattattttcacagattaacaccggagagaagggatcatgataaccctgtcagcagacagtttaattgcccacaccatggccaaaatgaaaagccgtctggaacaacgtaccggcgatgacgtttcacagctgcgcagtggtctgctgtttatggggaatattcaggatgcctatccccgtcgcctgttactggatgatcgtctgtcaccgctggataaaacgggctggatgatgatccgtttgtatgcgcaacaaaatgaaggggcggttttccccagctacgatgaattgcaggtgctgctggcttcaccctataaaggcaaagcctcccgtgaaaccgtcagccgggtattactgatgctgcgaataaccggctggctgagtttatgcaagcgtatccgcgatgagcacgggcgggtgcggggcaatatctatgcgcaacacgatgaaccgctgacctgccgtgatgccgagattcttgatctgcag

SEQ ID NO: 9 - DNA, 751 bp, Xenorhabdus nematophilus
taccaaccccgtcatgatttgctgcaacaaaagattattttagtcaccggtgccggtgatggcattgggcgtgaagtcgcactgacttatgcccgccacggggcgcaggtcattttgctggggcgtactgccgcaaaactggaagcagtcaaacaacaaattgaaaacgaagggggtttacccgccagcgtgtatacgatggatctcctgactgtcaccgcagaagagtgccagacttttgccaaagaactggcacagcgctattcctgtctggatggcgttttgcacaatgcagggcttcttggtgttgttgcaccggtcgtggaacaacctgtgcagctttggcatgatgtgatgcaagtgaatgtcaatgccacttttatgctgactcaggcattgattcccctgttattaaaatccgccagcggttctttagtctttaccagctccagcgtaggacgtgaaggccggagtggttggggagcatattctgtttccaaatttgccacagaaggctttatgcaggttttaacacaagaatatcaagacactgccctacgcattaattgcatcaatccggggggaacccgtaccaacatgcgtgccactgcgtttcccgatgagaactcagccaaattaaaaacacctgcggagattatgccagtctatctttatatcatgggtgatgacagccgaaatgattccggtatcagttttgatgctcaacccggtcgcaaagcggggcctgctg

SEQ ID NO: 10 - DNA, 596 bp, Xenorhabdus nematophilus
ttaccaatcaacgcctttttgtgcttttactccggcatcaaaagcatgttttaccggacgcatttcggtaatggtatcggcatattccagtaattctcggtgacatcctcgtccagtgatgatcacactctgatatgacgggcgctgtttgagtgccgtcagaacctcttcaagctccagataaccgtagctgaccatataagtcagttcatccagaacaacaagatttaatgaaggatcagacaacattgccaaggcatgtttccatgtggtttggccaccgcggtgtctgtttcacggttttgcgtctcccacgtaaaaccggttgccattatatgaaattcaacgccttgctgtgacagtaaatttctttcgccactttcccatgttcctttgataaattggataacgccggcgcgctgaccatgcccgattgctctggcaacagtaccgaatgccgccgtggtttttccttttccgttgccggtgaatacgataacaataccccgttcttgttgcgcagcttcaatacgggcatcgactttctctttcagacgttgctgtcttttttggtactggctgttatttctggtcat

SEQ ID NO: 11 - DNA, 20 bp, Artificial
  synthetic PCR primer
caacggtggtatatccagtg

SEQ ID NO: 12 - DNA, 21 bp, Artificial
  synthetic PCR primer
catatcaccagctcaccgtct

SEQ ID NO: 13 - DNA, 20 bp, Artificial
  synthetic PCR primer
ggttattgtctcatgagcgg

SEQ ID NO: 14 - DNA, 21 bp, Artificial
  synthetic PCR primer
tgcacccaactgatcttcagc

(19) United States
(12) Patent Application Publication
(10) Pub. No.: US 2005/0086715 A1
(43) Pub. Date: Apr. 21, 2005

(54) Xenorhabdus nematophila DNA sequences induced within insects

(76) Inventors: Heidi A. Goodrich-Blair, Madison, WI (US); Kimberly Cowles, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 10/965,566

(22) Filed: Oct. 14, 2004

Related U.S. Application Data
(60) Provisional application No. 60/511,879, filed on Oct. 16, 2003.

(51) Int. Cl. A01H 1/00; C12N 15/82; C07H 21/04; C07K 14/195; A01N 65/00; C12N 5/04

(52) U.S. Cl. 800/279; 435/69.1; 435/419; 435/468; 530/350; 514/12; 536/23.6

(57) ABSTRACT

Several DNA sequences of Xenorhabdus nematophila are disclosed. These DNA sequences are induced after the bacteria invade the body of a larvae insect of the order Lepidoptera. The DNA sequences can be used to obtain polypeptides that have insecticidal activities.

### CROSS-REFERENCE TO RELATED APPLICATIONS

This application claims the benefit of U.S. provisional application 60/511,879, filed on Oct. 16, 2003, which is incorporated by reference in its entirety.

### STATEMENT REGARDING FEDERALLY SPONSORED RESEARCH OR DEVELOPMENT

This invention was made with United States government support awarded by the following agency: USDA 00-CRHF-0-6055. The United States has certain rights in this invention.

### BACKGROUND OF THE INVENTION

Xenorhabdus nematophila is the gram-negative bacterial symbiont of Steinernema carpocapsae nematodes, which reproduce inside cadavers of larvae insects primarily of the order Lepidoptera. The life cycle of the nematode includes a non-feeding, free-living stage whose sole purpose is to locate and infect a new insect host. This infective stage carries a pure culture of Xenorhabdus nematophila bacteria within its intestine as it enters the blood system (hemocoel) of an insect host. The nematode then releases Xenorhabdus nematophila bacteria into the hemocoel by defecation. The insect host is then killed allowing the bacteria and the nematode to feed and multiply. Xenorhabdus nematophila is primarily responsible for the killing of the insect. It has been shown that Xenorhabdus nematophila can kill an insect host when injected directly into the hemocoel. When food becomes scarce in the insect cadaver, the nematodes reassociate with Xenorhabdus nematophila bacteria to get ready to infect other insect hosts.

Many larval lepidopterans that the nematode-bacterium complex infects and kills are agricultural pests. Thus, Xenorhabdus nematophila or a Steinernema carpocapsae-Xenorhabdus nematophila pair may be useful as biological pest control tools. Identification of Xenorhabdus nematophila proteins that are responsible for the killing can also provide new tools for biological control of pests. These new control tools are important and desirable given that many pesticides are losing their effectiveness in farmers' fields, and that pesticides are harmful to human health (e.g. linked to diseases such as prostate cancer). One factor that is involved in Xenorhabdus nematophila's killing of insects is an exotoxin produced by the bacteria. The exotoxin-encoding genes of Xenorhabdus nematophila have been cloned. However, the mode of action of these genes has not been extensively studied. It is not known whether or what additional factors are involved in Xenorhabdus nematophila's killing of insects.

In other bacteria such as Yersinia enterocolitica, Salmonella typhimurium, and Pseudomonas aeruginosa, many factors called virulence factors have been identified to contribute to host-damaging activities of the bacteria. The expression of these virulence factors is frequently induced upon the bacteria's entering into the host.

### BRIEF SUMMARY OF THE INVENTION

The present invention discloses several Xenorhabdus nematophila DNA sequences whose expression is induced specifically after the bacteria invade into the body of a larvae insect of the order Lepidoptera. These DNA sequences are useful for obtaining insecticidal genes and their protein products.

One aspect of the present invention relates to various polynucleotides, vectors and cells that comprise the DNA sequences of the invention or fragments thereof. Another aspect of the invention relates to the polypeptides encoded by the DNA sequences disclosed herein and antibodies to the polypeptides.

### BRIEF DESCRIPTION OF THE SEVERAL VIEWS OF THE DRAWINGS

FIG. 1 shows that a lesion in the iip 2 locus resulted in reduced bacterial virulence. Wild-type X. nematophila: ♦; iip 2 mutant X. nematophila: ▪.

### DETAILED DESCRIPTION OF THE INVENTION

This invention discloses Xenorhabdus nematophila DNA sequences (SEQ ID NO:1, SEQ ID NO:5, and SEQ ID NO:8) having open reading frames (ORFs) whose expression can be induced after the bacteria invade the body of a larval insect of the order Lepidoptera. The DNA sequences were obtained by genetically engineering a promoter-less chloramphenicol resistance gene into various places of the Xenorhabdus nematophila genome, injecting the resulting bacteria and chloramphenicol-succinate into a Manduca sexta larvae, selecting for bacteria that were resistant to chloramphenicol only in vivo but not in vitro, and sequencing the DNA sequences around the chloramphenicol resistance gene. As in many other bacteria such as Yersinia enterocolitica, Salmonella typhimurium, and Pseudomonas aeruginosa, loci of the Xenorhabdus nematophila genome that were specifically upregulated upon entry into a host are likely to represent pathogenesis genes (e.g., genes that provide the function of host defense evasion, host survival, and host killing). The ability to upregulate these genes is important for Xenorhabdus nematophila bacteria to survive and eventually kill an insect host. Thus, the DNA sequences disclosed here can be used to obtain genes and their protein products that have insecticidal activities. In addition, one can determine the active infection of larvae insects of the order Lepidoptera by Xenorhabdus nematophila bacteria through analyzing the expression of the ORFs in the insects' body.

The first DNA sequence is provided as SEQ ID NO:1 and termed iip2. SEQ ID NO:1 (iip2) contains three ORFs that appear to encode part of a nonribosomal peptide synthetase, which has a phosphopantetheine attachment site. The DNA sequences of the three ORFs are provided as SEQ ID NO:2, 3 and 4, respectively. The nonribosomal peptide synthetase encoded by iip2 is 44% identical and 60% similar to Anabaena strain 90 apdD, which is part of an operon required for the synthesis of an anabaenopeptilide (Rouhiainen et al., Mol. Microbiol. 37: 156, 2000). Anabaenopeptilides are similar to cyclic peptides that are toxic to mammals and plants due to inhibition of protein phosphatase 1 and 2A (MacKintosh et al., FEBS Lett. 264: 187-192, 1999; Yoshizawa et al., J. Cancer Res. Clin. Oncol. 116: 609, 1990).

The second DNA sequence is provided as SEQ ID NO:5 and termed iip4. SEQ ID NO:5 (iip4) contains an ORF (SEQ ID NO:6) for a putative membrane protein, an ORF (SEQ ID NO:7) for a putative lipoprotein, and other ORFs, which can be readily identified by a program such as OrfFinder available at “http://www.ncbi.nlm.nih.gov/gorf/gorf.html.” Lipoproteins as a protein class have been shown to have an important role in virulence.

The third DNA sequence is provided as SEQ ID NO:8 and termed iip6. SEQ ID NO:8 (iip6) contains an ORF (SEQ ID NO:9) for a putative dehydrogenase and an ORF (SEQ ID NO:10) with similarity to cobA of E. coli.

In one aspect, the present invention relates to an isolated polynucleotide comprising a nucleotide sequence or a complement of the nucleotide sequence wherein the nucleotide sequence is at least 80%, preferably at least 90%, more preferably at least 95%, and most preferably 100% identical to one of SEQ ID NO:1-10. In a preferred embodiment, the nucleotide sequence is at least 80%, at least 90%, at least 95%, or 100% identical to SEQ ID NO:2, SEQ ID NO:3, SEQ ID NO:4, SEQ ID NO:6, SEQ ID NO:7, SEQ ID NO:9, or SEQ ID NO:10. In another preferred embodiment, the nucleotide sequence is at least 80%, at least 90%, at least 95%, or 100% identical to SEQ ID NO:2, SEQ ID NO:3, SEQ ID NO:4, SEQ ID NO:7, or SEQ ID NO:9.

In another aspect, the present invention relates to an isolated polynucleotide comprising a nucleotide sequence or a complement of the nucleotide sequence wherein the nucleotide sequence is identical to at least 20, 25, 30, 35, 40, 45, 50, 100 or 150 continuous nucleotides of one of SEQ ID NO:1-10. In a preferred embodiment, the nucleotide sequence is identical to at least 20, 25, 30, 35, 40, 45, 50, 100 or 150 continuous nucleotides of one of SEQ ID NO:2, SEQ ID NO:3, SEQ ID NO:4, SEQ ID NO:6, SEQ ID NO:7, SEQ ID NO:9, and SEQ ID NO:10. In another preferred embodiment, the nucleotide sequence is identical to at least 20, 25, 30, 35, 40, 45, 50, 100 or 150 continuous nucleotides of one of SEQ ID NO:2, SEQ ID NO:3, SEQ ID NO:4, SEQ ID NO:7, and SEQ ID NO:9.

In another aspect, the present invention relates to a vector comprising any polynucleotide of the present invention described above in a manner known to those skilled in the art. The vector can be a cloning vector or an expression vector. In an expression vector, the polynucleotide is under the transcriptional control of one or more non-native expression control sequences, such as a promoter not natively found adjacent to the polynucleotide, such that an encoded polypeptide can be produced when the vector is provided in a compatible host cell or in a cell-free transcription and translation system. Such cell-based and cell-free systems are well known to the skilled artisan. Cells comprising a vector disclosed herein are themselves within the scope of the present invention.

In another aspect, the present invention relates to an isolated polypeptide that comprises an amino acid sequence encoded by SEQ ID NOs:2, 3, 4, 6, 7, 9 or 10. In a preferred embodiment, the amino acid sequence is encoded by SEQ ID NOs:2, 3, 4, 7, or 9. With the amino acid sequence provided, a skilled artisan can readily generate a monoclonal or polyclonal antibody against the polypeptide. Such an antibody is also within the scope of the present invention.

The term “isolated polynucleotide” or “isolated polypeptide” used in the specification and claims of the present invention means a polynucleotide or polypeptide isolated from its natural environment or prepared using synthetic methods such as those known to one of ordinary skill in the art. Complete purification is not required in either case. Amino acid and nucleotide sequences that flank a polypeptide or polynucleotide that occurs in nature can but need not be absent from the isolated form. The polypeptides and polynucleotides of the invention can be isolated and purified from normally associated material in conventional ways such that in the purified preparation the polypeptide or polynucleotide is the predominant species in the preparation. At the very least, the degree of purification is such that the extraneous material in the preparation does not interfere with use of the polypeptide or polynucleotide of the invention in the manner disclosed herein. The polypeptide or polynucleotide is preferably at least about 85% pure, more preferably at least about 95% pure, and most preferably at least about 99% pure.

Further, an isolated polynucleotide has a structure that is not identical to that of any naturally occurring polynucleotide or to that of any fragment of a naturally occurring genomic polynucleotide spanning more than three separate genes. An isolated polynucleotide also includes, without limitation, (a) a polynucleotide having a sequence of a naturally occurring genomic or extrachromosomal nucleic acid molecule but which is not flanked by the coding sequences that flank the sequence in its natural position; (b) a polynucleotide incorporated into a vector or into a prokaryote or eukaryote genome such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are polynucleotides present in mixtures of clones, e.g., as these occur in a DNA library such as a cDNA or genomic DNA library. An isolated polynucleotide can be modified or unmodified DNA or RNA, whether fully or partially single-stranded or double-stranded or even triple-stranded. A polynucleotide can be chemically or enzymatically modified and can include so-called non-standard bases such as inosine.

As used herein, “percent identity” between two polynucleotides is synonymous to “percent homology,” which is determined using the algorithm of Karlin and Altschul (Proc. Natl. Acad. Sci. USA 87:2264-2268, 1990), modified as in Karlin and Altschul (Proc. Natl. Acad. Sci. USA 90:5873-5877, 1993). Such an algorithm is incorporated into the NBLAST program of Altschul et al. (J. Mol. Biol. 215:403-410, 1990). BLAST nucleotide searches are performed with the NBLAST program, score=100, wordlength=12, to obtain nucleotide sequences homologous to a polynucleotide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective program (e.g., NBLAST) are used. See “http://www.ncbi.nlm.nih.gov.”

It is expected that minor sequence variations in SEQ ID NO:1-10 associated with nucleotide additions, deletions, and mutations, whether naturally occurring or introduced in vitro, would not interfere with the usefulness of these sequences. Therefore, the scope of the present invention is intended to encompass minor variations in the claimed sequences.

The invention will be more fully understood upon consideration of the following non-limiting examples.

### EXAMPLE 1

### Materials and Methods

Experimental organisms and growth conditions: Xenorhabdus nematophila ATCC 19061 (Amp^{r}) was used to make a chromosomal library for the in vivo expression technology (IVET) screen. An X. nematophila ATCC 19061 Tn10 (Cm^{r}) mutant (generated in the inventors' laboratory and designated D11) was used as a positive control for resistance to chloramphenicol within the insect. Escherichia coli SM10 (λpir) (14) was used as the cloning strain for the X. nematophila chromosomal library and was the donor for mating into X. nematophila. E. coli DH5α (λpir) (14) was used to clone fragments for sequencing of the insect-inducible regions. All Xenorhabdus strains were grown at 30° C. in Luria-Bertani (LB) broth (10) that had been kept in the dark or on LB agar plates supplemented with 0.1% pyruvate (18). E. coli strains were grown at 37° C. in LB broth or on LB agar plates. Antibiotics were used at the following concentrations: ampicillin (Amp), 75 μg/ml for X. nematophila, and 150 μg/ml for E. coli; chloramphenicol (Cm), 30 mg/ml; and streptomycin (Str), 25 mg/ml. Permanent stocks of X. nematophila strains were maintained at −80° C. supplemented with 10% Dimethyl Sulfoxide. Manduca sexta (tobacco hornworm) insect eggs were obtained from Dr. Walter Goodman (Dept. of Entomology, UW-Madison) and were reared as described (17).

Plasmids and DNA manipulations: pKJN102, a derivative of pGY2 (20), was used to construct the promoter trap library. pKJN102 contains a promoterless chloramphenicol acetyl transferase (cat) gene with a unique BglII site just upstream. In addition, pKJN102 carries a streptomycin resistance cassette (aad), mobilization functions, and an oriR6K. pKJN102 was constructed by deleting an ApaLI fragment containing the ampicillin resistance gene on pGY2. All DNA manipulations were performed according to manufacturers' protocols and standard procedures (12). DNA-modifying enzymes were acquired from Promega (Madison, Wis.), TaKaRa (Otsu, Japan), and Fermentas (Hanover, Md.). Plasmid DNA isolation kits were purchased from Qiagen (QIAprep Spin Miniprep Kit).

Construction of the promoter trap library: X. nematophila chromosomal DNA was partially digested with Sau3AI. Random genomic fragments ranging in size from 0.5-2.5 kb were cloned into the BglII site of pKJN102 directly upstream of the promoterless cat gene. Plasmid clones were transformed into E. coli SM10 (λpir) and plasmid maintenance was selected with Str. Pools of 100 clones were conjugated into X. nematophila (8) and exconjugants were selected on LB Str Amp plates. Because pKJN102 carries an origin of replication (oriR6K) that cannot be maintained in X. nematophila, only those X. nematophila strains that have successfully integrated the plasmid into the chromosome will be able to survive the selection. Exconjugant pools were resuspended in LB Str supplemented with 10% dimethyl sulfoxide, grown overnight at 30° C., and stored at −80° C.

Isolation of insect-inducible promoters: 36 pools of 100 clones each were tested for the presence of an insect-inducible promoter using the promoterless cat gene as a marker for gene expression. Prior to the screening procedure, each pool was inoculated into dark LB broth and incubated at 30° C. overnight. The pool culture was then subcultured 1:100 in fresh dark LB broth and incubated at 30° C. for a further 24 h. The 24-h cultures were washed twice in phosphate buffered saline (PBS) (12), and serial dilutions were made in order to determine the number of colony-forming units (CFU) per ml. Approximately 15,000 CFU per pool were injected into each of five fourth instar M. sexta larvae using a 30-gauge Hamilton syringe. As there are 100 clones per pool, each clone should be represented by approximately 150 CFU per injection (sufficient number of cells to kill an insect larvae of this size). At 1-h post-injection, insects were injected with Cm-succinate (10 ml of a 200 mg/ml stock; Sigma, St. Louis, Mo.). Injected insects were kept at 26° C. under a 16-h light/8-h dark cycle with a constant supply of food (gypsy moth wheat germ agar; ICN Biomedicals Inc., Aurora, Ohio) and monitored for death (lack of response to stimuli and dark green color) over a 72-h period. Bacteria were recovered from dead insects by extracting hemolymph with a syringe and dilution plating for isolated colonies on LB Str Amp. Individual colonies were then screened for Cm sensitivity by replica plating. Clones containing promoters that were specifically induced within the insect were resistant to the Cm injection while inside the insect (in vivo conditions) but were sensitive to Cm on LB agar plates (in vitro conditions). Clones that were identified to contain insect-inducible promoters upstream of the cat gene were designated insect-inducible promoter (iip) candidates and were tested individually for the ability to kill insects in the presence of Cm-succinate 1-h post-injection.

Determination of sequence flanking the plasmid insertion: Arbitrary PCR (4) was used to obtain sequence data upstream and downstream of pKJN102 insertions in the X. nematophila chromosome. Nested primers were used to sequence directly upstream of the cat gene (CatUp: 5° CAACGGTGGTATATCCAGTG 3′ (SEQ ID NO:11) and ArbCatUp: 5′ CATATCACCAGCTCACCGTCT 3′ (SEQ ID NO:12)) or downstream of the pKJN102 insertions (Arb102in: 5′ GGTTATTGTCTCATGAGCGG 3′ (SEQ ID NO:13) and Arb102out: 5′ TGCACCCAACTGATCTTCAGC 3′ (SEQ ID NO: 14)). Additional sequence information was obtained by cloning pKJN102-containing genomic fragments with several restriction enzymes, including SacII, KpnI, ScaI, and NdeI. Sequencing reactions were carried out using the ABI BigDye sequencing system (Applied Biosystems, Foster City, Calif.) and were cleaned with Autoseq columns (Amersham Pharmacia, Piscataway, N.J.). All sequencing reactions were sent to the UW-Madison Biotechnology Sequencing Center for analysis. Sequence data was compared to those in the database using the BLAST program (2).

Results

IVET results: 36 pools of 100 random cat fusions were injected into fourth instar M. sexta larvae (5 insects per pool) at approximately 15,000 CFU per pool. Cm-succinate was injected 1 h after injection of the fusion pool. Clones containing active promoters fused to the cat gene were expected to be resistant to the Cm injection and therefore kill the insect within 72 h post-injection. Inactive promoter fusions were expected to be inhibited by the Cm and therefore prevented from causing insect death. Bacteria recovered from dead insects (i.e. those bacteria carrying active promoter-cat fusions) were plated on LB Amp Str and screened for in vitro sensitivity to Cm. Those clones that showed sensitivity to Cm under in vitro conditions were designated insect-inducible promoters (iip) and were retested individually for the ability to kill insects in the presence of Cm-succinate. Of the 36 pools tested (3600 clones), 56 iip candidates were individually screened and 3 iip candidates were identified (SEQ ID NO:1, SEQ ID NO:5, and SEQ ID NO:8) as reproducibly able to kill insects in the presence of chloramphenicol but unable to grow on LB Cm³⁰ agar.

Sequence information for iip candidates:

iip-2 (SEQ ID NO:1): Sequencing data of iip-2 shows that the cat reporter is fused to a gene encoding a putative peptide synthetase, in an orientation consistent with this sequence being insect-induced. The predicted product of this ORF is similar to peptide synthetases from a variety of other bacteria, including ApdD (Anabaena sp.90; accession number 9715734) (44% identity and 60% similarity) and NosA (Nostoc sp. GSV224; accession number 6563397). Peptide synthetases are composed of multiple modules with each module responsible for the addition of one amino acid to the peptide chain (15). A single module commonly contains conserved domains involved in peptide synthesis, including phosphopantetheine binding domains, condensation domains, and adenylation domains (9). The sequenced region of iip-2 contains three ORFs, each representing one module containing all three conserved domains. Further sequencing information may reveal additional modules that we can use to predict the composition of the peptide synthetase product.

Peptide synthetase enzymes produce a wide range of products, including siderophores, antibiotics, and toxic peptides (15). For example, antibiotic production in Bacillus brevis (9), siderophore synthesis in Y. pestis (3, 11), and toxin production in Microcystis aeruginosa (6) all have been linked to peptide synthetase loci. In some cases, such as the Y. pestis siderophore yersiniabactin, disruption of the peptide synthetase leads to a defect in virulence (3). As Xenorhabdus nematophila is known to produce siderophores, antibiotics and toxins (7), the iip-2 region encoding a putative peptide synthetase was expected to play a role in the production of a virulence factor. As predicted, an X. nematophila strain carrying a mutation in iip-2 is reduced in virulence.

iip-4 (SEQ ID NO:5): The iip-4 chromosomal locus contains several ORFs, one with similarity to a putative membrane protein-encoding gene from Y. pestis (accession number 16122636) and another with similarity to a gene predicted to encode a putative lipoprotein from Y. pestis (accession number 16122635) (64% identity and 18% similarity). Although expression of either of these two genes could be driving expression of the cat gene, the reporter is fused to the 5′ end of the putative lipoprotein. Based on initial reverse transcriptase-PCR experiments, it appears as though the membrane protein and lipoprotein are not co-transcribed, suggesting that the lipoprotein is driving expression of the cat gene. Lipoproteins in the spirochete Borrelia burgdorferi are differentially expressed within its two hosts (mammals and ticks) (1, 16) and it is conceivable that a similar phenomenon may be occurring with X. nematophila while it is inside the insect.

Lipoproteins have been shown to have an important role in virulence. Shigella flexneri requires an outer membrane lipoprotein for activation of a type III secretion system and invasion of host cells (13), and studies of Y. enterocolitica have revealed a requirement for another outer membrane lipoprotein, NlpD, for virulence in mice (5). The iip-4 lipoprotein is predicted to be in the outer membrane (19) and expected to play a similar role in X. nematophila virulence towards insects.

iip-6 (SEQ ID NO:8): The iip-6 chromosomal locus contains two ORFs (SEQ ID NO:9 and 10), one (SEQ ID NO:9) with similarity to a gene encoding a putative short chain dehydrogenase in Y. pestis (accession number 16122443) (78% identity and 90% similarity) and the other (SEQ ID NO:10) with similarity to cobA of E. coli, encoding a putative cob(I)alamin adenosyltransferase (accession number 16129231). As the two ORFs have the potential to be co-transcribed, it is unclear which may be responsible for the expression of the cat gene within the insect. Sequencing data show the location of the iip-6::cat fusion within the putative short chain dehydrogenase.

### EXAMPLE 2

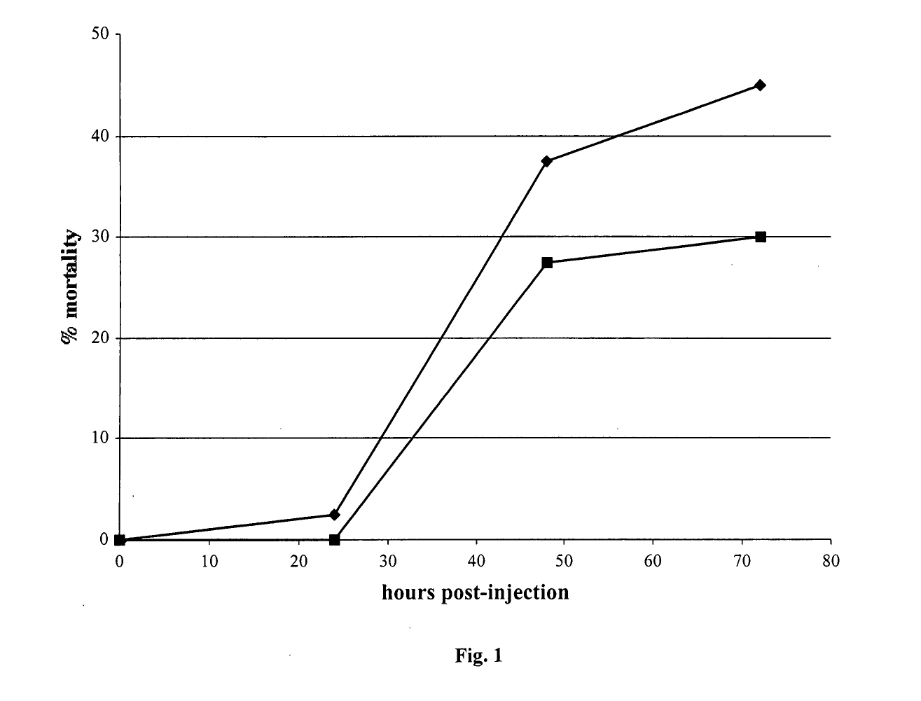

The inventors constructed an iip 2 mutant by inserting a kanamycin cassette into the non-ribosomal peptide synthetase located at this locus. The wild-type X. nematophila (HGB007) and the iip 2 mutant X. nematophila were injected into 20 insects (fourth instar Manduca sexta caterpillars) for each experiment. FIG. 1 summarizes the results of four independent experiments. As FIG. 1 shows, iip 2 mutants (black square line) demonstrated a reduced virulence phenotype, supporting the notion that iip 2 is important for bacterial virulence.

The present invention is not intended to be limited to the foregoing examples, but encompasses all such modifications and variations as come within the scope of the appended claims.

### REFERENCES

1. Akins, D. R., S. F. Porcella, T. G. Popova, D. Shevchenko, S. I. Baker, M. Li, M. V. Norgard, and J. D. Radolf. 1995. Evidence for in vivo but not in vitro expression of a Borrelia burgdorferi outer surface protein F (OspF) homologue. Mol. Microbiol. 18:507-520.
2. Altschul, S. F., T. L. Madden, A. A. Schaffer, J. Zhang, Z. Zhang, W. Miller, and D. J. Lipman. 1997. Gapped BLAST and PSI-BLAST: a new generation of protein database search programs. Nucleic Acids Research. 25:3389-3402.
3. Bearden, S. W., J. D. Fetherston, and R. D. Perry. 1997. Genetic organization of the yersiniabactin biosynthetic region and construction of avirulent mutants in Yersinia pestis. Infect. Immun. 65:1659-1668.
4. Caetano-Annoles, G. 1993. Amplifying DNA with arbitrary oligonucleotide primers. PCR Methods and Applications. 3:85-92.
5. Darwin, A. J., and V. L. Miller. 1999. Identification of Yersinia enterocolitica genes affecting survival in an animal host using signature-tagged transposon mutagenesis. Mol. Microbiol. 32:51-62.
6. Dittmann, E., B. A. Neilan, M. Erhard, H. von Dohren, and T. Borner. 1997. Insertional mutagenesis of a peptide synthetase gene that is responsible for hepatotoxin production in the cyanobacterium Microcystis aeruginosa PCC 7806. Mol. Microbiol. 26:779-787.
7. Forst, S., and K. Nealson. 1996. Molecular biology of the symbiotic-pathogenic bacteria Xenorhabdus spp. and Photorhabdus spp. Microbiol. Rev. 60:21-43.
8. Forst, S. A., and N. Tabatabai. 1997. Role of the histidine kinase, EnvZ, in the production of outer membrane proteins in the symbiotic-pathogenic bacterium Xenorhabdus nematophila. Appl. Environ. Microbiol. 63:962-968.
9. Kleinkauf, H., and H. von Dohren. 1990. Antibiotics—cloning of biosynthetic pathways. FEBS Letters. 268:405-7.
10. Miller, J. H. 1972. Experiments in Molecular Genetics. Cold Spring Harbor Laboratory Press, Cold Spring Harbor, N. Y.
11. Pelludat, C., A. Rakin, C. A. Jacobi, S. Schubert, and J. Heesemann. 1998. The yersiniabactin biosynthetic gene cluster of Yersinia enterocolitica: organization and siderophore-dependent regulation. J. Bacteriol. 180:538-546.
12. Sambrook, J., E. F. Fritsch, and T. Maniatis. 1989. Molecular Cloning: a Laboratory Manual, second ed. Cold Spring Harbor Laboratory Press, Cold Spring Harbor, N.Y.
13. Schuch, R., and A. T. Maurelli. 1999. The mxi-Spa type III secretory pathway of Shigella flexneri requires an outer membrane lipoprotein, MxiM, for invasin translocation. Infection & Immunity. 67:1982-1991.
14. Simon, R., U. Priefer, and A. Pühler. 1983. A broad host range mobilization system for in vivo genetic engineering: transposon mutagenesis in gram negative bacteria. Biotechnol. 1:784-791.
15. Stachelhaus, T., and M. A. Marahiel. 1995. Modular structure of genes encoding multifunctional peptide synthetases required for non-ribosomal peptide synthesis. FEMS Microbiol. Lett. 125:3-14.
16. Suk, K., S. Das, W. Sun, B. Jwang, S. W. Barthold, R. A. Flavell, and E. Fikrig. 1995. Borrelia burgdorferi genes selectively expressed in the infected host. Proc. Natl. Acad. Sci. USA. 92:4269-4273.
17. Vivas, E. I., and H. Goodrich-Blair. 2001. Xenorhabdus nematophila as a model for host-bacterium interactions: rpoS is necessary for mutualism with nematodes. J. Bacteriol. 183:4687-4693.
18. Xu, J., and R. E. Hurlbert. 1990. Toxicity of irradiated media for Xenorhabdus spp. Appl. Environ. Microbiol. 56:815-818.
19. Yamaguchi, K., F. Yu, and M. Inouye. 1988. A single amino acid determinant of the membrane localization of lipoproteins in E. coli. Cell. 53:423-432.
20. Young, G. M., D. Amid, and V. L. Miller. 1996. A biofunctional urease enhances survival of pathogenic Yersinia enterocolitica and Morganella morganii at low pH. J. Bacteriol. 178:6487-6495.

1. An isolated polynucleotide comprising a member selected from (a) SEQ ID NO:2, (b) SEQ ID NO:3, (c) SEQ ID NO:4, (d) SEQ ID NO:6, (e) SEQ ID NO:7, (f) SEQ ID NO:9, (g) SEQ ID NO:10, (h) a nucleotide sequence that is at least 80% identical to one of (a) through (g), or (i) a nucleotide sequence that is complementary to one of (a) through (h).

2. The isolated polynucleotide of claim 1, wherein the polynucleotide comprises a member selected from (a) SEQ ID NO:2, (b) SEQ ID NO:3, (c) SEQ ID NO:4, (d) SEQ ID NO:7, (e) SEQ ID NO:9, (f) a nucleotide sequence that is at least 80% identical to one of (a) through (e), or (g) a nucleotide sequence that is complementary to one of (a) through (f).

3. The isolated polynucleotide of claim 1, wherein the nucleotide sequence in (h) is at least 90% identical to one of (a) through (g).

4. The isolated polynucleotide of claim 1, wherein the polynucleotide comprises a member selected from SEQ ID NO:1, SEQ ID NO:5, SEQ ID NO:8, a nucleotide sequence that is at least 80% identical to any of the foregoing, or a complement of any of the foregoing.

5. A vector comprising the polynucleotide molecule of claim 1.

6. A host cell comprising the vector of claim 5.

7. An isolated polynucleotide comprising a member selected from (a) a nucleotide sequence that is identical to at least 20 contiguous nucleotides of SEQ ID NO:2, SEQ ID NO:3, SEQ ID NO:4, SEQ ID NO:6, SEQ ID NO:7, SEQ ID NO:9, or SEQ ID NO:10 or (b) a nucleotide sequence that is complementary to (a).

8. The isolated polynucleotide of claim 7, wherein the polynucleotide comprises a member selected from (a) a nucleotide sequence that is identical to at least 20 contiguous nucleotides of SEQ ID NO:2, SEQ ID NO:3, SEQ ID NO:4, SEQ ID NO:7, or SEQ ID NO:9 or (b) a nucleotide sequence that is complementary to (a).

9. The isolated polynucleotide of claim 7, wherein the nucleotide sequence in (a) is identical to at least 25 contiguous nucleotides of SEQ ID NO:2, SEQ ID NO:3, SEQ ID NO:4, SEQ ID NO:6, SEQ ID NO:7, SEQ ID NO:9, or SEQ ID NO:10.

10. The isolated polynucleotide of claim 7, wherein the nucleotide sequence in (a) is identical to at least 30 contiguous nucleotides of SEQ ID NO:2, SEQ ID NO:3, SEQ ID NO:4, SEQ ID NO:6, SEQ ID NO:7, SEQ ID NO:9, or SEQ ID NO:10.

11. The isolated polynucleotide of claim 7, wherein the nucleotide sequence in (a) is identical to at least 40 contiguous nucleotides of SEQ ID NO:2, SEQ ID NO:3, SEQ ID NO:4, SEQ ID NO:6, SEQ ID NO:7, SEQ ID NO:9, or SEQ ID NO:10.

12. The isolated polynucleotide of claim 7, wherein the nucleotide sequence in (a) is identical to at least 50 contiguous nucleotides of SEQ ID NO:2, SEQ ID NO:3, SEQ ID NO:4, SEQ ID NO:6, SEQ ID NO:7, SEQ ID NO:9, or SEQ ID NO:10.

13. The isolated polynucleotide of claim 7, wherein the nucleotide sequence in (a) is identical to at least 100 contiguous nucleotides of SEQ ID NO:2, SEQ ID NO:3, SEQ ID NO:4, SEQ ID NO:6, SEQ ID NO:7, SEQ ID NO:9, or SEQ ID NO:10.

14. An isolated polypeptide comprising an amino acid sequence encoded by a nucleotide sequence selected from SEQ ID NO:2, SEQ ID NO:3, SEQ ID NO:4, SEQ ID NO:6, SEQ ID NO:7, SEQ ID NO:9 or SEQ ID NO:10.

15. The isolated polypeptide of claim 14, wherein the polypeptide comprises an amino acid sequence encoded by a nucleotide sequence selected from SEQ ID NO:2, SEQ ID NO:3, SEQ ID NO:4, SEQ ID NO:7, or SEQ ID NO:9.

16. An antibody that can bind to a polypeptide consisting of an amino acid sequence encoded by a nucleotide sequence selected from SEQ ID NO:2, SEQ ID NO:3, SEQ ID NO:4, SEQ ID NO:6, SEQ ID NO:7, SEQ ID NO:9 or SEQ ID NO:10.
